(19) United States
(12) Patent Application Publication
(10) Pub. No.: US 2011/0124589 A1
(43) Pub. Date: May 26, 2011

(54) INHALED FOSFOMYCIN/TOBRAMYCIN FOR THE TREATMENT OF CHRONIC OBSTRUCTIVE PULMONARY DISEASE

(75) Inventors: Elizabeth Peters Bhatt, Belmont, CA (US); David Lloyd MacLeod, Issaquah, WA (US); Matthew Thomas McKevitt, Seattle, WA (US); Terry Glenn Newcomb, Seattle, WA (US)

(73) Assignee: Gilead Sciences, Inc., Foser City, CA (US)

(21) Appl. No.: 12/943,778

(22) Filed: Nov. 10, 2010

Related U.S. Application Data
(60) Provisional application No. 61/264,160, filed on Nov. 24, 2009.

(51) Int. Cl. A61K 31/7036; A61P 11/00; A61P 31/04; A61P 29/00

(52) U.S. Cl. 514/37

(57) ABSTRACT

The present invention provides the use of an aerosol formulation comprising fosfomycin and tobramycin in the treatment of patients with chronic obstructive pulmonary disease (COPD) who are experiencing or are at risk of experiencing acute exacerbations of COPD. Formulations for such use and methods of treating humans with COPD are also provided.

### CROSS-REFERENCE TO RELATED APPLICATIONS

This application claims priority to U.S. Provisional Patent Application Ser. No. 61/264,160 filed 24 Nov. 2009.

### FIELD OF THE INVENTION

The present invention relates to an inhaled composition containing a combination of fosfomycin and tobramycin for the treatment of patients who have Chronic Obstructive Pulmonary Disease (COPD) and who are experiencing or at risk of experiencing acute exacerbation, and methods for treating the same.

### BACKGROUND OF THE INVENTION

Chronic obstructive pulmonary disease (COPD), a smoking-related condition characterized by progressive and poorly reversible airflow obstruction and airway inflammation, is the fourth most common cause of death in developed countries. COPD is projected to be the third leading cause of global deaths in 2020 and is the only one of the four most common causes of death with an increasing mortality rate. In 2008 in the United States, there were an estimated 10 million patients diagnosed with chronic obstructive pulmonary disease (COPD). SDI COPD Claims Analysis, May 2009. Murray et al., 1997 Lancet 349: 1269-76. Approximately 7 million U.S. patients receive treatment for COPD. Mannino et al; The Epidemiology and Economics of COPD, Proc Am Thorac Soc 2007. In the US, direct COPD costs in 2002 were approximately $18.0 billion. Statistics from National Center for Health Statistics, National Health Interview Survey: Research for the 1995-2004 redesign, Hyattsville, Md.: U.S. Department of Health and Human Services, CDC, NCHS. Vital and Health Stat 2(126), 1999.

The clinical course of COPD is characterized by chronic disability, with intermittent, acute exacerbations which may be triggered by a variety of stimuli including exposure to pathogens, inhaled irritants (e.g., cigarette smoke), allergens, or pollutants. “Acute exacerbation” refers to worsening of a patient's COPD symptoms from his or her usual state that is beyond normal day-to-day variations, and is acute in onset. See, Rabe et al., 2007 Am J Res Crit Care Med, 176: 532-555. Acute exacerbations of COPD greatly affect the health and quality of life of patients with COPD. Bathoorn, E, Int J Chron Obstruct Pulmon Dis. 2008 3(2):217-229. Acute exacerbation of COPD is a key driver of the associated substantial socioeconomic costs of the disease. Approximately 73% ($13 billion) of direct COPD costs in 2002 were due to hospitalizations related to acute exacerbations of COPD. Investigators from the Burden of Obstructive Lung Disease (BOLD) Initiative have estimated the cumulative discounted cost of COPD care in the US to be $880 billion by 2020—an average of more than $44 billion per year over two decades. Lee et al., 2006 ATS Proceedings, 3:A598. Multiple studies have also shown that prior exacerbation is an independent risk factor for future hospitalization for COPD. Garcia-Aymerich et al., 2003, Thorax, 58:100-105. Hospitalization consumes roughly 70% of COPD healthcare expenditure in the US. McGhan et al., 2007, Chest, 132(6):1748-1755. Accordingly, for a new drug therapy to significantly reduce the health and economic costs of COPD, it must address acute exacerbations of COPD.

In the PULSE Study, Bayer examined the effects of oral moxifloxacin in 1157 patients with baseline FEV₁=70% predicted and FEV₁/FVC ratio <0.7, and 2 or more acute exacerbations of COPD in the last twelve months. Patients were randomized to oral moxifloxacin 400 mg daily for 5 days every 8 weeks or matched placebo, for a total of 48 weeks. The primary endpoint was reduction in number of acute exacerbations. Treatment with moxifloxacin was associated with a relative 45% reduction in the odds of exacerbation for the subgroup of patients who produced mucopurulent or purulent sputum in their stable baseline state. Sethi et al, 2010 Resp Res, 11:10.

In a Phase 2 study by MPex, MP-376, an aerosol formulation of levofloxacin, was evaluated for prevention of acute exacerbations in COPD. The study was completed in April 2010, enrolling and randomizing roughly 300 patients to MP-376, administered for 5 days every 28 days, or matching placebo, for 6 months. http://clinicaltrials.gov/ct2/show/NCT00739648 August 2010. Enrolled patients have moderate-to-severe COPD with a history of 2 or more acute exacerbations in the previous year. Study results have not yet been reported.

U.S. Patent Application Publication No. US2009/0054374 to Paringenix Inc. relates to methods for treating and preventing acute exacerbations of COPD comprising administering O-desulfated heparin intravenously.

PCT Publication No. WO2005/110022 to Gilead Sciences, Inc. discloses a fosfomycin plus tobramycin combination formulation for delivery by aerosolization. The fosfomycin/tobramycin combination formulation containing an efficacious amount of fosfomycin and tobramycin is able to inhibit susceptible bacteria. Fosfomycin and tobramycin are formulated in solution such that when reconstituted, the pH is between 4.5 and 8.0 or as a dry powder. Also disclosed is a method for treatment of respiratory tract infections by a formulation delivered as an aerosol having mass medium aerodynamic diameter predominantly from 1 to 5 microns, produced by a jet or ultrasonic nebulizer (or equivalent) or dry powder inhaler.

### SUMMARY OF THE INVENTION

As a first aspect, the invention provides a method for treating a human with chronic obstructive pulmonary disease (COPD) who is experiencing or at risk of experiencing an acute exacerbation of COPD. The method comprises administering by inhalation to the human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

As another aspect, the invention provides a method for reducing the frequency, severity or duration of an acute exacerbation in a human with COPD. The method comprises administering by inhalation to the human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

As another aspect, the invention provides a method for treating one or more symptoms of an acute exacerbation in a human with COPD. The method comprises administering by inhalation to the human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

As another aspect, the invention provides a method for reducing the frequency, severity or duration of one or more symptoms of an acute exacerbation in a human with COPD. The method comprises administering by inhalation to the human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

As another aspect, the invention provides a method of treating a bacterial infection in the respiratory tract of a human by administering by inhalation to the human an aerosol formulation consisting of fosfomycin and tobramycin and optionally one or more pharmaceutically acceptable carriers, excipients and/or diluents, wherein the weight ratio is from about 7 to about 9 parts by weight of fosfomycin to from about 1 to about 3 parts by weight of tobramycin, wherein said formulation is suitable for administration by a nebulizer, dry powder inhaler or metered dose inhaler the improvement comprising reducing the frequency, severity or duration of an acute exacerbation in a human with chronic obstructive pulmonary disease.

As another aspect, the present invention provides a method of reducing pulmonary inflammation in a human with COPD. The method comprises administering by inhalation to the human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

In one embodiment, the methods of the invention utilize an aerosol formulation containing 4 parts by weight of fosfomycin and 1 parts by weight of tobramycin.

In another aspect, the invention provides the use of an aerosol formulation comprising fosfomycin and tobramycin wherein the weight ratio is from about 7 to about 9 parts by weight of fosfomycin to from about 1 to about 3 parts by weight of tobramycin, in the manufacture of a medicine suitable for administration by inhalation, for treating a human with COPD who is experiencing or at risk of experiencing acute exacerbation of COPD.

In another aspect, the invention provides the use of an aerosol formulation comprising fosfomycin and tobramycin wherein the weight ratio is from about 7 to about 9 parts by weight of fosfomycin to from about 1 to about 3 parts by weight of tobramycin, in the manufacture of a medicine suitable for administration by inhalation, for reducing the frequency, severity or duration of acute exacerbation in a human with COPD.

In another aspect, the invention provides the use of an aerosol formulation comprising fosfomycin and tobramycin wherein the weight ratio is from about 7 to about 9 parts by weight of fosfomycin to from about 1 to about 3 parts by weight of tobramycin, in the manufacture of a medicine suitable for administration by inhalation, for treating one or more symptoms of acute exacerbation in a human with COPD.

In another aspect, the invention provides the use of an aerosol formulation comprising fosfomycin and tobramycin wherein the weight ratio is from about 7 to about 9 parts by weight of fosfomycin to from about 1 to about 3 parts by weight of tobramycin, in the manufacture of a medicine suitable for administration by inhalation, for reducing the frequency, severity or duration of one or more symptoms of acute exacerbation in a human with COPD.

In another aspect, the invention provides the use of an aerosol formulation consisting of fosfomycin and tobramycin and optionally one or more pharmaceutically acceptable carriers, excipients and/or diluents, in a physiologically acceptable solution wherein the weight ratio is from about 7 to about 9 parts by weight of fosfomycin to from about 1 to about 3 parts by weight of tobramycin, in the manufacture of a medicine suitable for administration by a nebulizer, dry powder inhaler or metered dose inhaler, for treating a bacterial infection in the respiratory tract of a human,

the improvement comprising
reducing the frequency, severity or duration of acute exacerbation in a human with chronic obstructive pulmonary disease.

### BRIEF DESCRIPTION OF THE DRAWINGS

FIG. 1: Time-kill curves for a 9:1 fosfomycin:tobramycin combination against Pseudomonas aeruginosa ATCC 27853 evaluated in the presence of 2% mucin. Symbols: Δ no drug control, ▴ fosfomycin (14.4 μg/mL), tobramycin (1.6 μg/mL), [symbol] fosfomycin (14.4 μg/mL) tobramycin (1.6 μg/mL), and --- bactericidal line.

FIG. 2: Time-kill curves for a 4:1 fosfomycin:tobramycin combination against P. aeruginosa ATCC 27853 evaluated in the presence of 2% mucin. Symbols: Δ no drug control, ▴ fosfomycin (12.8 μg/mL), tobramycin (3.2 μg/mL), [symbol] fosfomycin (12.8 μg/mL)+tobramycin (3.2 μg/mL), and --- bactericidal line.

FIG. 3: Time-kill curves for a 7:3 fosfomycin:tobramycin combination against P. aeruginosa ATCC 27853 evaluated in the presence of 2% mucin. Symbols: Δ no drug control, ▴ fosfomycin (11.2 μg/mL), tobramycin (4.8 μg/mL), [symbol] fosfomycin (11.2 μg/mL)+tobramycin (4.8 μg/mL), and --- bactericidal line.

FIG. 4: Fosfomycin Time-Kill Curves for P. aeruginosa evaluated in the presence of 2% mucin. Symbols: (▴) no drug control, (□) 4 μg/mL, ( [symbol] ) 8 μg/mL, (◯) 16 μg/mL, () 32 μg/mL, (---) bactericidal line.

FIG. 5: Tobramycin Time-Kill Curves for P. aeruginosa evaluated in the presence of 2% mucin. Symbols: (▴) no drug control, (□) 0.5 μg/mL, (¦) 1 μg/mL, (◯) 2 μg/mL, () 4 μg/mL, (---) bactericidal line.

FIG. 6: FTI Time-Kill Curves for P. aeruginosa evaluated in the presence of 2% mucin. Symbols: (□) no drug control, (▴) 4 μg/mL FTI, () 8 μg/mL FTI, ( [symbol] ) 16 μg/mL FTI, (A) 32 μg/mL FTI, (---) bactericidal line. The FTI concentrations reflect the sum of the individual component concentrations in a 4:1 ratio (e.g., 8 μg/mL FTI=6.4 μg/mL fosfomycin+1.6 μg/mL tobramycin).

FIG. 7: Effects of FTI, Fosfomycin, and Tobramycin on P. aeruginosa Protein Synthesis. Symbols: (♦) 8 μg/mL FTI (6.4 μg/mL fosfomycin+1.6 μg/mL tobramycin), ( [symbol] ) 6.4 μg/mL fosfomycin, (▴) 1.6 μg/mL tobramycin FIG. 8: Effects of FTI, Fosfomycin, and Tobramycin on P. aeruginosa Cell Wall Synthesis. Symbols: (♦) 8 μg/mL FTI (6.4 μg/mL fosfomycin+1.6 μg/mL tobramycin), ( [symbol] ) 6.4 μg/mL fosfomycin, (♦) 1.6 μg/mL tobramycin.

FIG. 9: Effect of Fosfomycin on Bacterial Uptake of Tobramycin.

FIG. 10: Reduction of P. aeruginosa C177 CFU in the Rat Lung After Intratracheal Administration of 0.1, 1, 2.5, 5 and 10 mg/kg of FTI Antibiotic was administered twice daily for 3 days. Averages and standard deviations are shown. *P<0.05, **P<0.01.

FIG. 11: Reduction of P. aeruginosa (strain C177) CFU in the rat lung after intratracheal administration of 0.1, 0.5, 1, and 2.5 mg/kg of tobramycin.

FIG. 12: Reduction of P. aeruginosa (strain 0177) CFU in the rat lung after intratracheal administration of 1, 2.5, 5, and 10 mg/kg of fosfomycin.

### DETAILED DESCRIPTION OF THE INVENTION

As used herein:
“FTI” refers to an aerosol formulation of fosfomycin and tobramycin which is suitable for administration by inhalation.
“9:1 fosfomycin:tobramycin” and “9:1 Fos:Tob” are synonymous and mean a liquid or dry powder pharmaceutical formulation containing a 9:1 ratio by weight of fosfomycin acid to tobramycin base.
“4:1 fosfomycin:tobramycin” and “4:1 Fos:Tob” are synonymous and mean a liquid or dry powder pharmaceutical formulation containing a 4:1 ratio by weight of fosfomycin acid to tobramycin base such that the amount of fosfomycin is four times the amount of tobramycin (by weight).
“7:3 fosfomycin:tobramycin” or “7:3 Fos:Tob” are synonymous and mean a liquid or dry powder pharmaceutical formulation containing a 7:3 ratio by weight of fosfomycin acid to tobramycin base.
“5:5 fosfomycin:tobramycin” or “5:5 Fos:Tob” are synonymous and mean a liquid or dry powder pharmaceutical formulation containing a 50:50 ratio by weight of fosfomycin acid to tobramycin base.
“COPD” refers to chronic obstructive pulmonary disease as defined by GOLD (see, Background) and is treated herein as inclusive of the same disease known by the alternative expressions “chronic obstructive respiratory disease” (CORD), “chronic obstructive airways diseases” (COAD), “chronic obstruction lung disease” (COLD), and “chronic airway limitation” (CAL).
“Acute exacerbation(s)” and “acute exacerbations in humans with COPD” are synonymous and refer to worsening of a patient's COPD symptoms from his or her usual state, that is beyond normal day-to-day variations, and is acute in onset.
“Acute exacerbations of chronic bronchitis in humans with COPD” refers to worsening of a COPD patient's chronic bronchitis symptoms from his or her usual state, that is beyond normal day-to-day variations and is acute in onset. Chronic bronchitis symptoms include dyspnea, excessive cough, sputum production, sputum purulence, change in color of sputum, chest tightness, reduced exercise tolerance, and fatigue
“Acute bacterial exacerbations of chronic bronchitis in patients with COPD” refers to a clinical diagnosis of presumptive bacterial infection superimposed on a chronic pulmonary condition. The term is defined by the FDA Center for Drug Evaluation and Research (CDER) in the Guidance for Industry on “Acute Bacterial Exacerbations of Chronic Bronchitis in Patients with COPD: Developing Antimicrobial Drugs for Treatment,” August 2008, Clinical Antimicrobial Division, Revision 1. According to the FDA Guidance, acute bacterial exacerbations of chronic bronchitis in patients with COPD may be described as bronchial inflammation associated with the isolation of pathogenic bacteria from sputum or bronchial lavage specimens. The role of bacteria is complicated in acute exacerbations as chronic bacterial colonization may be present in the airways of patients with COPD. Latent bacterial infection may also contribute to persistent inflammation.
“Frequent exacerbator” refers to a human who suffers from or is undergoing treatment for COPD and who experiences at least 2, and more typically 3 or more, acute exacerbations during a 12 month period.
“FEV₁” refers to forced expiratory volume in 1 second and is a typical objective measure of a patient's respiratory condition.
“FEV₁/FVC” refers to FEV₁/forced vital capacity.
“Minimal inhibitory concentration (MIC)” means the lowest concentration of antibiotic (s) that prevents visible growth after incubation for 18-20 hours at 35° C.
“Minimal bactericidal concentration (MBC)” means the lowest concentration of antibiotic that results in =3 Log₁₀ of bacterial killing.
“Time-dependent killing” refers to an antibiotic in which the essential pharmacodynamic parameter is the time that drug concentrations remain above the MIC such that drug concentrations higher than the MIC do not kill bacteria any faster or to a greater extent.
“Concentration-dependent killing” refers to antibiotics in which the essential pharmacodynamic parameter is the drug concentration, such that the higher the drug concentration achieved, the greater the rate and extent of bacterial killing.
“Bacteriostatic” means the antibiotic acts by inhibiting bacterial growth.
“Bactericidal” means the antibiotics acts by killing bacteria.

### Acute Exacerbations of COPD

COPD is defined by the Global Initiative for Chronic Obstructive Lung Disease (GOLD) as “a disease state characterized by airflow limitation that is not fully reversible. The airflow limitation is usually both progressive and associated with an abnormal inflammatory response of the lungs to noxious particles or gases.” R A Pauwels et al., 2001 Am J Respir Crit Care Med 163:1256-1276. Airflow limitation is the slowing of expiratory airflow as measured by spirometry, with a persistently low forced expiratory volume in 1 second (FEV₁). The FEV₁ percent predicted is used to divide patients into 4 grades of severity. The GOLD definition of airflow limitation is an FEV₁/FVC ratio of less than 70%. Id.

Previously, COPD was characterized by the classic Venn diagram depicting COPD at the intersection of three overlapping disease entities: chronic bronchitis, emphysema, and asthma. Chronic bronchitis is clinically defined as excessive cough and sputum production on most days for at least three months during at least two consecutive years. Emphysema is characterized by chronic dyspnea (shortness of breath) resulting from destruction of lung tissue and enlargement of air spaces, and expiratory flow limitation. Bronchiectasis is an abnormal stretching and enlarging of respiratory passages caused by a cycle of infection, inflammation and tissue damage in the airways. Asthma is an inflammatory disease of lung airways that makes the airways prone to constrict too much and too easily in response to stimuli. Asthma differs from COPD in that the loss of pulmonary function in asthma is reversible. The GOLD definition of COPD does not differentiate between chronic bronchitis and emphysema but does note that although asthma and COPD can coexist, the largely reversible airflow limitation in asthma merits different therapeutic approaches than the largely irreversible airflow limitation of COPD. Mannimo, Hospital Physician October 2001 22-31.

Common symptoms of COPD include dyspnea, sputum, coughing, upper airway symptoms such as colds and sore throats, wheezing, chest tightness, fatigue, fluid retention, and acute confusion. An acute exacerbation of COPD is typically a noticeable change from the COPD patient's baseline, typical or day-to-day condition. Thus, acute exacerbations may manifest as worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, acute confusion, and combinations of any two or more of these symptoms.

In patients who have the most frequent episodes acute exacerbations can be a major determinant of disease progression. Acute exacerbation of COPD has been shown to be an independent predictor of mortality, with risk of mortality correlated with frequency of exacerbations. Soler-Cataluna, 2005 Thorax, 60: 925-931. Acute exacerbations may accelerate lung function decline and account for approximately 25% of the FEV₁ decline in COPD patients. Seemungal et al., 2000 Am J Res Crit Care Med 161:1608-1613. Symptoms and lung function may take several weeks to recover to baseline following acute exacerbation. Id. In moderate-severe patients, 22% had a recurrent event within 50 days due to persistently high systemic inflammation. Perera et la., 2007 Eur Res J29:527. In one study, patients with moderate-to-severe COPD with >2.92 exacerbations per year experienced a 25% greater decline in FEV₁ in one year compared to patients with <2.92 exacerbations per year. Donaldson et al., 2002, Thorax 57: 847-852.

Recent data indicate that persistent bacterial airway infection develops at the earliest stages of COPD in roughly 30% of patients. Monso et al., 1999 European Respir J 13:338-42. This may be facilitated by smoke-related damage to innate lung defenses such as mucociliary clearance and the epithelial barrier. Curtis et al., 2007 PATS 4:512-521. Moreover, patients susceptible to frequent exacerbations appear to have significantly higher levels of inflammatory markers in induced sputum as well as latent bronchial infections. One study found exacerbation frequency to be significantly related to latent bronchial infection (p=0.023) while bacterial load in the stable state was found to be significantly correlated with sputum IL-8 levels (P=0.02). Patel et al., 2002, Thorax 57:759-764. Accordingly, it is currently believed that anti-infective drug therapy may reduce acute exacerbations in COPD in frequent exacerbators.

Common causes of acute exacerbations include inflammation, particularly chronic inflammation, infection, including chronic or persistent infection, pollution and allergens. Among the pathogenic triggers, 24% are believed to be viral, 30% bacterial and 25% both viral and bacterial. Papi 2006 Amer J Respir Crit Care Med 173:114-121. Viral pathogens associated with acute exacerbations in patients with COPD include rhinoviruses, influenza, parainfluenza, coronavirus, adenovirus, and respiratory syncytial virus.

Sethi and Murphy 2008 NEJM 359(22):2355-2365, reviewed the bacterial species typically responsible for acute exacerbations of COPD. According to their findings, Haemophilus influenzae (Gram-negative) occurs in 20-30% of exacerbations; Streptococcus pneumoniae (Gram-positive) and Moraxella catarrhalis (Gram-negative) each occur in 10-15% of exacerbations and P. aeruginosa (Gram-negative) occurs in 5-10% of exacerbations. Id. Chlamydophila pneumoniae (Gram-negative) and Mycoplasma pneumoniae contribute to 1-5% of the exacerbations. Id. Additionally, Legionella pneumoniae (Gram-negative) may be another etiologic agent. Id. Other bacterial species that may play a role (albeit less frequently) include Haemophilus haemolyticus (Gram-negative), Haemophilus parainfluenzae (Gram-negative), Enterobacter species (Gram-negative) and Staphylococus aureus (Gram-positive).

In August 2008, the FDA published an industry guide for program development and the design of clinical trials for antimicrobial drug products for the treatment of acute bacterial exacerbations of chronic bronchitis in patients with CORD (ABECB-COPD). FDA Center for Drug Evaluation and Research (CDER) in the Guidance for Industry on “Acute Bacterial Exacerbations of Chronic Bronchitis in Patients with COPD: Developing Antimicrobial Drugs for Treatment,” August 2008, Clinical Antimicrobial Division, Revision 1. According to that guidance, the pathogens most commonly associated with acute exacerbations in patients with COPD are S. pneumoniae, H. influenzae, and M. catarrhalis, and as such the goal of ABECB-COPD clinical trials should be to demonstrate an effect of antibacterial therapy on the clinical course of ABECB-COPD presumptively associated with these species.

The rate of bacterial infection in stable COPD increases with worsening airflow obstruction. The highest rate of persistent bacterial infection has been identified in stable patients with moderate-to-severe COPD who experience ≧2.6 exacerbations per year. Patel et al., 2002 Thorax 57:759-764. Bacterial infection is implicated in COPD pathogenesis and progression, by providing the inflammatory stimulus to both 1) dysregulate the host inflammatory response during the stable disease state, and 2) serve as an inflammatory trigger for acute exacerbations.

While the bacterial pathogens in the lower airways of stable COPD patients do not cause acute symptoms of infection at early stages of disease, multiple studies have shown that their presence is associated with inflammatory and immune responses characteristic of COPD. For this reason, the presence of bacterial pathogens in the lower airways may be characterized as latent bronchial infection. Monso, 2004 Arch Bronconeumol 40(12):543-6. In one study comparing infected and noninfected patients (n=26) with no differences in age, pack-years of smoking, or spirometry, recovery of a bacterial pathogen by BAL was associated with significant elevation of neutrophils, IL-8, and the proteinase MMP-9. Sethi et al., 2006, Am J Res Crit. Care Med 173:991-998. Evidence of persistent bronchial infection in COPD has also been obtained in histologic and radiographic studies. One seminal, histologic study showed the presence of lymphoid follicles composed of B and T lymphocytes in the small airways of patients with severe COPD, suggesting an acquired immune response to persistent bacterial infection. Hogg et al., 2004, NEJM 350:2645-53. Also, the high prevalence of bronchiectasis in patients with moderate-to-severe COPD (shown in a recent study to be present in 50% of patients (n=54), see, Patel et al., 2004 Am J Res Crit Care Med 170:400-407) suggests high rates of chronic persistent infection. In fact, potential pathogens were identified in over 50% of patients, with P. aeruginosa present in 17.9%.

Though it may be latent, bacterial infection of lower airways in stable COPD does not appear to be innocuous. Multiple studies suggest that bacterial infection in stable COPD has a significant impact on COPD progression, which in all cases is associated with pro-inflammatory effects. In a recent study of stable patients (n=30) with moderate-to-severe COPD, sputum bacterial load was shown to be significantly associated with increased IL-8 levels and accelerated decline in FEV₁. Wilkinson et al., 2003 Am J Res Crit Care Med 167:1090-1095. Sequential sampling in this study showed a significantly greater FEV₁ decline in patients with a change in the infecting pathogen over the course of the study, compared with patients infected with the same bacterial pathogen, suggesting that latent bronchial infection is a dynamic process resulting in enhanced inflammatory effects.

One aforementioned study found exacerbation frequency to be significantly related to latent bronchial infection (p=0.023) and bacterial load in the stable state to be significantly correlated with sputum IL-8 levels (P=0.02). Patel et al., 2002 Thorax 57:759-764. An association of infection with non-typeable H. influenzae in the stable state was also found, with increased total symptoms and sputum purulence during exacerbation, as well as longer time to recovery of peak flow after an exacerbation. The implication of these results is that latent bronchial infection may be an important cause of acute exacerbations by serving as an exogenous stimulus to chronic airway inflammation. Despite the actual trigger for an exacerbation, this study suggests that latent infection with certain bacterial pathogens (in this case non-typeable H. influenzae) may predispose to more severe, bacterial exacerbations. Id.

Chronic inflammation also plays a central role in COPD pathogenesis and progression. Research has helped characterize the chronic airway inflammatory response—as well as an independent systemic inflammatory response which may have a role in muscle wasting and other significant extrapulmonary co-morbidities of COPD. Creutzberg et al., 2000 Am J Res Crit Care Med 161:745-752. While asthma and COPD are both recognized as inflammatory diseases, the profile of the airway inflammation in COPD and asthma is radically different. Asthma is typically characterized by mucosal infiltration of eosinophils, increased Th2 lymphocytes and activated mast cells. In COPD there is a predominance of neutrophils, macrophages, and both Th1 and Tc1 cells, with relative greater number of the latter. Barnes et al., 2003 Eur Res J 22:672-688. Inflammatory mediators demonstrate a corresponding pattern, with prominence of neutrophil chemoattractants such as LTB₄, IL-8 and TNF-alpha in COPD. Acute exacerbation is associated with further increase in these inflammatory mediators, as well as an associated increase in NF-kappaB activation in alveolar macrophages. Aaron et al., 2001 Amer J Respir Crit Care Med 163:349-55 and Caramori et al., 2003 Thorax 58:348-351. The pathophysiologic consequences include mucus hypersecretion and mucosal edema secondary to increased neutrophil degranulation, and direct (primarily LTB₄-related) increase in bronchial tone. Nadel et al., 2000, Chest, 17: 386S-95S and Gompertz et al., 2001, ERJ, 17: 1112-9. In combination, these changes lead to the worsening airflow limitation and dynamic hyperinflation characteristic of acute exacerbation. O'Donnell et al., 2006, Thorax, 61:354-61.

In addition to creating an environment prone to acute exacerbations, the inflammatory cascade induces inflammatory factors thought to be responsible for damage to the lung tissue. Most of these deleterious factors are released by neutrophils, such as serine proteinases, elastase, and proteinase 3—all of which are known to cause emphysema. It appears that the increased levels of these factors during exacerbations correspond with periods of accelerated tissue damage. A recent study suggests that neutrophilic airway inflammation is dramatically induced in all COPD exacerbations, regardless of whether the etiology of exacerbation is the consequence of a pathogenic trigger. A. Papi, et al., 2006 Am J Resp Crit Care Med 173:1114-1121.

COPD patients who experience a minimum of 2, or generally 3, acute exacerbations of COPD annually are referred to as “frequent exacerbators.” More than 1 million patients in the US—the majority of patients who have exacerbations—would qualify as frequent exacerbators. Anzeuto et al., 2009 Am J Res Crit Care Med 179:A1527. Persistent bacterial infection may be a significant factor in COPD patients who experience frequent exacerbations. Combination viral and bacterial infections are more severe and require longer hospitalizations. Hurst et al., 2005 European Respir J 26:846-852 and Seemungal et al., 2001 Amer J Respir Crit Care Med 164:1618-1623.

The particular COPD phenotype of frequent exacerbators is of growing interest at least partly due to research showing that COPD patients who experience ≧2.6 exacerbations per year do not simply have more severe underlying COPD. Perara et al. 2007 Eur Res J 29:527-534. What frequent exacerbators do appear to have in common is increased airway inflammation: one clinical study of patients with moderate-to-severe COPD (n=57) showed that patients with 3 or more exacerbations per year had significantly higher levels of induced sputum IL-6 and IL-8 in the steady state than patients with 2 or less exacerbations per year; there was no correlation between these inflammatory markers and baseline lung function. Bhowmik et al., 2000 Thorax 55: 114-120).

Acute exacerbations, regardless of their trigger, are typically treated with increased bronchodilation, systemic corticosteroids and/or oral antibiotics. Current therapies including inhaled corticosteroids, long acting beta agonists and long acting muscarinic antagonists have shown a 20-25% decrease in exacerbations in long-term studies. An estimated 60-88% of patients that have exacerbations are treated with antibiotics. Adelphi COPD DSP VII 2008 and Adams et al., 2000 Chest 117:1345-1352. Unfortunately, there is no single antibiotic of choice for treatment of exacerbations in COPD and long-term effects are a concern particularly in the prevalence of antibiotic resistance.

Treatment of acute exacerbations in moderate-to-severe COPD patients with antibiotics and systemic corticosteroids has proven beneficial, particularly when purulent sputum is present. A recent Cochraine review found reduction in mortality and treatment failure with treatment of moderate-to severe-exacerbations, as well as likely reduction in exacerbation duration. Ram et al., 2006, Cochrane Database Syst Rev, 2:CD004403. Community-based studies of antibiotics did not demonstrate benefit, particularly when used in mild exacerbations. Allegra et al., Pulm Pharmacol Ther 2001 14:49-55. However a placebo-controlled study of amoxicillin/clavulate not included in this meta-analysis did show better resolution of symptoms compared with placebo, with greater benefit seen with increasing severity of disease. Allegra et al., Pulm Pharmacol Ther 2001 supra).

A retrospective observational study of roughly 19,000 Dutch patients with moderate COPD, compared treatment of acute exacerbations with oral antibiotics and corticosteroids versus oral corticosteroids alone. In that study, the combination therapy resulted in significantly increased time to next exacerbation, and reduced mortality, compared to treatment with corticosteroids alone. B M Roede et al., 2008 Thorax 63(11):968-973. The comparative time between second and third exacerbations for these two groups was 240 days versus 127 days (p<0.001). Exposure to antibiotics between exacerbations was also associated with a lower risk of subsequent acute exacerbations.

Shorter courses of oral antibiotic therapy for acute exacerbations may be desirable. A recent meta-analysis of studies comparing antibiotic courses longer than 5 days with courses less than 5 days found no difference in efficacy. El Moussaoui et al., 2008 Thorax 63:415-422.

Reducing the frequency, severity and duration of exacerbations is a key unmet need in the treatment of COPD patients. The overall effect of frequent acute exacerbations in patients with COPD contributes to more rapid reduction in quality of life, morbidity, mortality, and healthcare costs. It is currently believed that patients with COPD who are suffering from or susceptible to acute exacerbations, particularly moderate-to-severe COPD patients who are frequent exacerbators, will benefit from treatment with antibiotics, particularly in an antibiotic regimen involving intermittent course of short duration. This strategy targets both the bacterial pathogens that have latently infected lower airways—potentially reducing underlying airway inflammation and subsequent risk of acute exacerbations—as well as any new bacterial pathogens (or strains of pathogens) that may trigger an acute exacerbation.

Antibiotics may offer an additional advantage over anti-inflammatory agents, particularly inhaled corticosteroids, in the treatment of COPD, in that antibiotic therapy may target the upstream stimulus to the inflammatory cascade characteristic of COPD and thereby potentially avoid the pitfalls of redundant inflammatory pathways. Furthermore, antibiotics would not disable appropriate host-mediated immune response to pathogens. While inhaled corticosteroids have been shown (with and without long-acting beta₂ agonists) to reduce incidence of acute exacerbations, they are also associated with increased risk of pneumonia. See, Calverley et al., 2007, NEJM, 356:775-89.

### Methods of Treatment and Uses

Generally, the present invention provides methods of treating humans with COPD. “Treating” and “treatment”, as used herein refer to reversing, alleviating, inhibiting the progress of, or preventing the disorder or condition or one or more symptoms of the disorder or condition. In particular embodiments of the present invention, “treating” refers to treating an acute exacerbation of COPD, reducing the frequency, duration or severity of an acute exacerbation of COPD, treating one or more symptoms of acute exacerbation of COPD, reducing the frequency, duration or severity of one or more symptoms of an acute exacerbation of COPD, preventing the incidence of acute exacerbation of COPD, or preventing the incidence of one or more symptoms of acute exacerbation of COPD, in a human. The reduction in frequency, duration or severity is relative to the frequency, duration or severity of an acute exacerbation or symptom in the same human not undergoing treatment according to the methods of the present invention. A reduction in frequency, duration or severity of acute exacerbation or one or more symptoms of acute exacerbation may be measured by clinical observation by an ordinarily skilled clinician with experience treating COPD patients or by subjective self evaluations by the patient undergoing treatment. Clinical observations by an ordinarily skilled clinician may include objective measures of lung function such as FEV₁ or FEV₁/FVC, as well as the frequency with which intervention is required to maintain the patient in his or her most stable condition, and the frequency of hospital admission and length of hospital stay required to maintain the patient in his or her most stable condition.

Typically, subjective self evaluations by a patient are collected using industry-recognized and/or FDA-recognized patient reported outcome (PRO) tools. Such tools may allow the patient to evaluate specific symptoms or other subjective measures of quality of life. An example of one patient reported outcome tool is Exacerbations from Pulmonary Disease Tool (EXACT-PRO), which is currently being developed for evaluating clinical response in acute bacterial exacerbations of chronic bronchitis in patients with COPD by United BioSource Corporation along with a consortium of pharmaceutical industry sponsors in consultation with the FDA.

The symptoms of acute exacerbation include worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, acute confusion, and combinations of any two or more of these symptoms. Not all of the foregoing symptoms are required for a worsening of the COPD patient's condition to be identified as acute exacerbation. Acute exacerbations may manifest in the form of a subset of these symptoms. Accordingly, the inventors contemplate the practice of the inventive methods wherein only a subset of the foregoing symptoms of acute exacerbation are present.

In the methods and uses of the present invention, the “human with COPD” is a human who suffers from or is undergoing treatment for COPD and is either experiencing an acute exacerbation of COPD or at risk of experiencing an acute exacerbation of COPD. In one embodiment, the “human with COPD” is a human who has experienced at least one acute exacerbation of COPD in the past 24 months. In one particular embodiment, the “human with COPD” is a human who has experienced at least one acute exacerbation of COPD in the past 12 months. In one embodiment, the “human with COPD” is a frequent exacerbator.

In one embodiment, the present invention provides a method of treating a human with COPD who is experiencing or at risk of experiencing an acute exacerbation of COPD. In one embodiment, the present invention provides a method of treating a human with COPD who is experiencing or at risk of experiencing an acute exacerbation of COPD manifested by one or more symptoms selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, or any subset thereof.

In one embodiment, the present invention provides methods for reducing the frequency, duration and/or severity of acute exacerbation of COPD in a human. In one embodiment, the present invention provides a method of reducing the frequency, duration and/or severity of acute exacerbation of COPD manifested by one or more symptoms selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, or any subset thereof.

In another embodiment, the invention provides methods for treating one or more symptoms of acute exacerbation of COPD in a human. In one particular embodiment, the invention provides methods for treating one or more symptoms of acute exacerbation of COPD in a human, wherein the symptoms are selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, or any subset thereof.

In another embodiment, the invention provides methods for reducing the frequency, duration and/or severity of any one or more symptoms of acute exacerbation of CORD in a human, wherein the symptoms are selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, or any subset thereof.

In one embodiment, the invention provides a method of treating a bacterial infection in the respiratory tract of a human by administering by inhalation to the human an aerosol formulation consisting of fosfomycin and tobramycin in a physiologically acceptable solution wherein the weight ratio is from about 7 to about 9 parts by weight of fosfomycin to from about 1 to about 3 parts by weight of tobramycin, wherein the formulation is suitable for administration by a nebulizer, dry powder inhaler or metered dose inhaler, the improvement comprising reducing the frequency, severity or duration of an acute exacerbation in a human with CORD. In one embodiment, the weight ratio is about 9 parts fosfomycin to about 1 parts tobramycin. In one embodiment, the weight ratio is about 4 parts fosfomycin to about 1 parts tobramycin. In one embodiment, the weight ratio is about 7 parts fosfomycin to about 3 parts tobramycin.

In another embodiment, the present invention provides a method of reducing pulmonary inflammation in a human with COPD. The method comprises administering by inhalation to the human an aerosol formulation comprising an effective amount of a combination of fosfomycin and tobramycin, wherein the weight ratio is from about 7 to about 9 parts by weight of fosfomycin to from about 1 to about 3 parts by weight of tobramycin. In one embodiment, the weight ratio is about 9 parts fosfomycin to about 1 parts tobramycin. In one embodiment, the weight ratio is about 4 parts fosfomycin to about 1 parts tobramycin. In one embodiment, the weight ratio is about 7 parts fosfomycin to about 3 parts tobramycin. Reducing pulmonary inflammation according to the methods of the present invention may have the effect of reducing destruction of airway tissue as well as improving lung function and reducing the frequency, duration and severity of acute exacerbations (or symptoms thereof) in patients with COPD.

In another embodiment, the invention provides, the use of an aerosol formulation comprising fosfomycin and tobramycin, in the manufacture of a medicine suitable for administration by inhalation, for treating a human with COPD who is experiencing or at risk of experiencing an acute exacerbation of COPD. In another embodiment, the invention provides, the use of an aerosol formulation comprising fosfomycin and tobramycin, in the manufacture of a medicine suitable for administration by inhalation, for reducing the frequency, severity or duration of an acute exacerbation of COPD in a human. In one particular embodiment, the acute exacerbation of COPD is manifested by one or more symptoms selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, or any subset thereof, and the method comprises reducing the frequency, severity or duration of one or more of said symptoms.

In one embodiment, the invention provides, the use of an aerosol formulation comprising fosfomycin and tobramycin in the manufacture of a medicine suitable for administration by inhalation, for treating one or more symptoms of an acute exacerbation of COPD in a human. In another embodiment, the invention provides, the use of an aerosol formulation comprising fosfomycin and tobramycin in the manufacture of a medicine suitable for administration by inhalation, for reducing the frequency, severity or duration of one or more symptoms of an acute exacerbation of COPD in a human. In one particular embodiment, the one or more symptoms are selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, or any subset thereof. In one embodiment, the weight ratio is about 9 parts fosfomycin to about 1 parts tobramycin. In one embodiment, the weight ratio is about 4 parts fosfomycin to about 1 parts tobramycin. In one embodiment, the weight ratio is about 7 parts fosfomycin to about 3 parts tobramycin.

The methods and uses of the present invention all comprise the step of administering by inhalation to the human, an aerosol formulation containing an effective amount of a combination of fosfomycin and tobramycin.

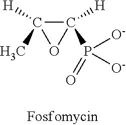

is a broad spectrum phosphonic acid antibiotic. Kahan, F. M., et al., 1974 Ann NY Aced Sci 253:364-386, and Woodruff et al., 1977 Chemother 23(1):1-22. Fosfomycin that has bactericidal activity against Gram-negative bacteria including Citrobacter spp., Escherichia coli, Enterobacter spp., Klebsiella pneumoniae, P. aeruginosa, Salmonella spp., Shigella spp., and Serratia marcescens as well as Gram-positive bacteria including vancomycin resistant enterococci, methicillin-resistant S. aureus (MRSA), methicillin-sensitive S. aureus (MSSA), and S. pneumoniae. Greenwood et. al., 1992 Infection 20(4):S305-S309; Grimm, 1979 Infect 7(4):256-259; Marchese et. al., 2003 Int Antimicrob Agents 22(2):53-59; and Schulin, 2002 J Antimicrob Chemother 49:403-406; and Perri et. al., 2002 Diagn Microbiol Infect Dis 42:269-271. Fosfomycin has the greatest activity against E. coli, Proteus spp., Salmonella spp., Shigella spp., and S. marcescens which are generally inhibited at fosfomycin concentrations=64 μg/mL (Forsgren and Walder, 1983 “Antimicrobial activity of fosfomycin in vitro” J Antimicrob Chemother 11(5):467-471). Fosfomycin is moderately active against P. aeruginosa (Forsgren and Welder, supra), particularly when compared to tobramycin (Schulin, supra). Fosfomycin is bactericidal but exhibits time-dependent killing against E. coli and S. aureus (Grif et al., 2001 J Antimicrob Chemother 48:209-217). The rate and degree of killing depends on the length of time fosfomycin is in contact with the target organism (Craig, 1998 Clin infect Dis 26 (1):1-12; Mueller et al., 2004 Antimicrob Agents Chemother 48(2):369-377). Increasing the fosfomycin concentration will not produce a corresponding increase in the rate or degree of killing activity. This feature is significant because it is preferable to treat P. aeruginosa infections with antibiotics that exhibit bactericidal, concentration-dependent killing activity (Craig and Mueller et. al., supra).

Fosfomycin is widely distributed in various body tissues and fluids but does not significantly bind to plasma proteins. Consequently, fosfomycin is available to exert antibacterial effects if it reaches sufficient concentrations at the site of infection.

Fosfomycin inhibits the first step of peptidoglycan biosynthesis in the bacterial cell wall. F M Kahan, et al., 1974 Ann NY Acad Sci 235:364-386 and H B Woodruff, et al., 1977 Chemotherapy 23(Suppl 1):1-22. Fosfomycin has a high mutation frequency resulting in bacterial resistance in vitro. J L Martinez, et al., 2000Antimicrob Agent Chemother 44:1771-1777 and Nilsson et al., 2003 Antimicrob Agents Chemother 47(9):2850-2858. When fosfomycin resistance occurs, it is typically due to a genetic mutation in one or both of the chromosomally encoded transport systems, and less commonly by modifying enzymes. Arca et al., 1997 J Antimicrob Chemother 40:393-399; and Nilsson at al., 2003, supra.

Fosfomycin is commercially available as fosfomycin disodium, fosfomycin trometamol and fosfomycin calcium. Both fosfomycin calcium and fosfomycin trometamol are oral formulations while fosfomycin disodium is an intravenous formulation. Only oral fosfomycin trometamol is approved in the USA for treating uncomplicated urinary tract infections. An aerosol formulation deliverable directly to the lungs is not yet commercially available.

The methods of the present invention may employ any form of fosfomycin, with the choice of the particular form of fosfomycin being well within the discretion of one skilled in the art. Fosfomycin disodium is currently a preferred form the preparation of aerosol formulations designed for administration as a solution for nebulization or by dry powder inhalation via a metered dose inhaler or dry powder inhaler.

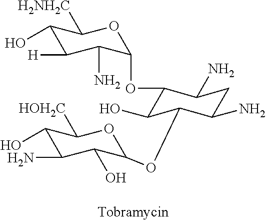

is an aminoglycoside antibiotic that is active against Gram-negative aerobic bacilli including P. aeruginosa, E. coli, Acinetobacter spp., Citrobacter spp., Enterobacter spp., K. pneumoniae, Proteus spp., Salmonella spp., S. marcescens, and Shigella spp (Vakulenko et al., 2003 Clin Microbial Rev 16(3):430-450). In particular, tobramycin is highly active against P. aeruginosa. The tobramycin MICs of susceptible P. aeruginosa are typically less than 2 μg/ml. (Shawar et al., 1999 Antimicrob Agents Chemother 43(12):2877-2880; Spencker et al., 2002 Clin Microbial Infect 9:370-379; and Van Eldere, 2003 J Antimicrob Chemother 51:347-352). Most Gram-positive bacteria are resistant to tobramycin, with the exception of S. aureus and S. epidermidis (Vakulenko, et al., supra).

Tobramycin is rapidly bactericidal and acts by inhibiting bacterial protein synthesis. Tobramycin must traverse the cytoplasmic membrane prior to interacting with the ribosome and initiating bactericidal effects. Tobramycin exhibits concentration-dependent killing. Increasing the tobramycin concentration increases both the rate and extent of bacterial killing. Therefore, to achieve therapeutic success, it is necessary to administer a large enough dose to produce a peak tobramycin level 5-10 times greater than the MIC of the target organism at the site of infection. It is preferable to treat P. aeruginosa infections with antibiotics that exhibit bactericidal, concentration-dependent killing activity (Ansorg et al., 1990 Chemother 36:222-229).

Tobramycin is usually administered to treat less serious Gram-negative bacterial infections (Vakulenko, et al., supra). However, it may be combined with other classes of antibiotics to treat severe infections of the urinary tract and abdomen, as well as endocarditis and bacteremia (Id.). Parenteral administration of tobramycin in combination with cell-wall inhibiting antibiotics has been used to treat respiratory infections, in particular those caused by P. aeruginosa in CF patients.

Tobramycin is poorly absorbed orally and must be administered parenterally. Tobramycin is available in both intravenous and aerosol formulations. After parenteral administration, tobramycin is primarily distributed within the extracellular fluid. Tobramycin is rapidly excreted by glomular filtration resulting in a plasma half-life of 1-2 hours. Tan et al., 2003 Am J Respir Crit Care Med 167(6):819-823. Penetration of tobramycin into respiratory secretions is very poor and its activity is further reduced by binding to sputum (Kuhn, 2001 Chest 120:94 S-98S). Aerosol administration of tobramycin results in significantly higher sputum levels of =1000 μg/mL (Geller et al., 2002 Chest 122:219-226) compared with intravenous administration, but sputum binding remains a significant problem. The respiratory tract of COPD patients are commonly obstructed with sputum. The effectiveness of several classes of antibiotics such as aminoglycosides and β-lactams is reduced due to poor penetration into sputum and the activity of these antibiotics is further reduced by binding to sputum components. See, Hunt et al., 1995 Antimicrob Agents Chemother 39(1):34-39; Kuhn, 2001 Chest 120:94 S-98S; Ramphal et al., 1988 J Antimicrob Chemother 22:483-490; and Mendelman et al., 1985 Am Rev Respir Dis 132(4):761-765.

Bacterial resistance to tobramycin has become increasingly prevalent due to repeated and prolonged antibiotic monotherapy. Conway et al, 2003 Am J Respir Med 2(4):321-332; Van Eldere, 2003 J Antimicrob Chemother 51:347-352; Mirakhur et al., 2003 J Cyst fibros 2(1):19-24; Pitt et al., 2005 Thorax 58(9):794-796; Schulin, 2002 J Antimicrob Chemother 49:403-404 CF patients are colonized with P. aeruginosa strains which are largely resistant to tobramycin gentamicin, ceftazidinme, piperacillin, and ciprofloxacin. Thus, existing antibiotic therapies are becoming ineffective for treating P. aeruginosa respiratory infections because of drug resistance.

In the US, the most widely used aerosolized antibiotic for treatment of CF patients is tobramycin inhalation solution (TIS), which has been shown to produce substantial improvements in pulmonary function and other clinical parameters in CF patients. TIS has been available in the US for over 10 years, however, some clinicians are reluctant to use aerosolized tobramycin for chronic suppressive therapy in patients with respiratory infections fearing that long-term exposure could further promote resistance and diminish the effectiveness of intravenous aminoglycoside therapy. In order to reduce the risk of treatment-emergent resistance, TIS treatment in CF patients has been restricted to alternating courses of 28 days on drug followed by 28 days off drug. Aminoglycosides also have nephrotoxic and ototoxic effects that require routine monitoring of serum concentrations when administered intravenously. Aminoglycoside toxicity is cumulative and consequently, repeated administration by any route raises concern about total lifetime exposure to these agents.

As a consequence of the concern over toxicity, it would be beneficial to provide a liquid or dry powder formulation combining fosfomycin and tobramycin wherein tobramycin is present in minimal amounts which are effective when combined with fosfomycin, and which can be efficiently administered by dry powder inhalation or nebulization solution.

Tobramycin is commercially available as a base or sulfate salt. Either form is suitable for use in the methods and formulations of the present invention. Conveniently, tobramycin base is commercially available as a dry powder which may be used as such for dry powder inhalation formulations or reconstituted with a pharmaceutically acceptable diluent for solution formulations for nebulization. Both Tobramycin base and tobramycin sulphate are preferred forms for use in the methods, therapeutic uses and formulations of the present invention.

### Formulation

The aerosol formulation utilized in the methods and uses of the present invention contains a combination of fosfomycin and tobramycin. It is currently believed that the combination of fosfomycin and tobramycin in FTI offers certain advantages over other conventional antibiotics. FTI is active against important COPD respiratory pathogens including P. aeruginosa (including multidrug resistant P. aeruginosa) S. aureus, H. influenzae, M. catarrhalis, and Enterobacteriaceae. FTI is rapidly bactericidal and has activity comparable to tobramycin. Moreover, FTI has been shown to reduce the development of antibiotic resistance. Fosfomycin, the major component of FTI, has a very favorable safety profile when administered parenterally. Since tobramycin constitutes between 30% and 10%, preferably 20%, of FTI on a weight basis, the cumulative toxic effects due to tobramycin could also be reduced.

The major component of FTI, fosfomycin, inhibits the first step of peptidoglycan biosynthesis in the bacterial cell wall by irreversibly binding to the enzyme phosphoenolpyruvate (UDP-N-acetylglucosamine enolpyruval-transferase). The minor component, tobramycin, prevents protein biosynthesis by causing translational errors and by inhibiting translocation. Based on these mechanisms of action, evidence is provided that suggest the enhanced activities of FTI are due to increased uptake of tobramycin: (i) time-kill curves demonstrate that both FTI and tobramycin are bactericidal and killed in a concentration-dependent fashion despite the fact that fosfomycin, the major component, was only bacteriostatic and killed in a time-dependent fashion (ii) macromolecular biosynthesis studies showed that FTI and tobramycin inhibited protein synthesis in a concentration-dependent fashion (iii) FTI inhibited protein synthesis faster and to a greater degree than cell wall biosynthesis at a concentration (8 μg/ml) and in a time-frame (2-4 h) that corresponded to bactericidal killing (iv) addition of 10 μg/mL fosfomycin resulted in a 170% increase in ³H-tobramycin uptake relative to the no-fosfomycin control. The precise identity of the protein(s) that facilitate the energy-dependent transport of tobramycin across the inner membrane are currently unknown.

### A. Ratio of Components

The aerosol formulation contains a combination of from about 7 to about 9 parts by weight of fosfomycin and from about 1 to about 3 parts by weight of tobramycin. More particularly, the ratio of components in the combination is about 9:1, about 4:1 or about 7:3. In one embodiment, the aerosol formulation contains no antibiotic agents other than fosfomycin and tobramycin. In one embodiment, the aerosol formulation is a liquid or solution formulation containing no active agents other than fosfomycin, tobramycin and optionally saline, such as hypertonic saline. In one embodiment, the aerosol formulation is a dry powder containing no active agents other than fosfomycin and tobramycin.

The combinations of fosfomycin and tobramycin in weight ratios of about 91, about 4:1 and about 7:3 (fosfomycin:tobramycin (“Fos:Tob”)) were evaluated for in vitro antimicrobial activity, killing rates, and frequency of resistance. This data was published in PCT Application No. WO2005/11022 to Gilead Sciences Inc., (formerly Corus Pharma) and in D L MacLeod et al., “Antibacterial activities of fosfomycin/tob-ramycin combination: a novel inhaled antibiotic for bronchiectasis,” 2009 J Antimicrob Chemother 64(4) 829-836 (hereinafter “MacLeod, 2009 JAC”) Advance Access published 13 Aug. 2009. Certain data is also repeated in the Examples below.

In the chequerboard synergy study previously reported (PCT Application No. WO2005/11022), interactions between fosfomycin and tobramycin were determined by the broth microdilution chequerboard method. Id. Synergy was defined as an FICI of =0.5, no interaction as an FICI >0.5 and =4, and antagonism as an FICI >4. The lowest FICI was used for final interpretation of drug interactions. No antagonism was seen between fosfomycin and tobramycin for any of the 27 strains tested: S. aureus, n=4; P. aeruginosa, n=17; E. coli, n=5; and H. influenzae, n=1. Id. The combination was categorized as no interaction for 25 of the 27 strains (93%) and synergistic for 1 P. aeruginosa strain and 1 E. coli strain. Id. Thus, chequerboard synergy studies failed to demonstrate synergistic properties of the combination which are evident from time-kill studies as reported in the examples below.

Minimum inhibitory concentration studies and time-kill studies demonstrated that all three combinations of fosfomycin and tobramycin were active against common respiratory pathogens. The 7:3 and 4:1 Fos:Tob combinations showed superior bactericidal activity relative to the 9:1 combination. It is desirable for long-term toxicity reasons to reduce the chronic exposure to tobramycin. Accordingly, in one preferred embodiment, the formulation contains a combination of about 4 parts by weight of fosfomycin and about 1 parts by weight of tobramycin.

### B. Aerosol Formulations and Delivery Devices

The aerosol formulation according to the present invention is a pharmaceutical composition. In one aspect, the invention provides aerosol formulations comprising a combination of fosfomycin and tobramycin and optionally one or more pharmaceutically acceptable excipients, diluents or carriers or combinations thereof. The pharmaceutically acceptable excipient(s), diluent(s) or carrier(s) must be acceptable in the sense of being compatible with the other ingredients of the aerosol formulation and not deleterious to the recipient thereof. Generally, the pharmaceutically acceptable excipient(s), diluent(s) or carrier(s) employed in the pharmaceutical formulation are “non-toxic” meaning that it/they is/are deemed safe for consumption in the amount delivered in the aerosol formulation and “inert” meaning that it/they does/do not appreciable react with or result in an undesired effect on the therapeutic activity of the active ingredients (fosfomycin and tobramycin). Pharmaceutically acceptable excipients, diluents and carriers are conventional in the art and may be selected using conventional techniques, based upon the desired route of administration. See, REMINGTON'S, PHARMACEUTICAL SCIENCES, Lippincott Williams & Wilkins; 21^{st} Ed (May 1, 2005). Preferably, the pharmaceutically acceptable excipient(s), diluent(s) or carrier(s) are Generally Regarded As Safe (GRAS) according to the FDA.

Dry powder compositions for topical delivery to the lung endobronchial space by inhalation may be formulated without excipient or carrier and instead including only the active ingredients in a dry powder form having a suitable particle size for inhalation. Dry powder compositions may also contain a mix of the active ingredient and a suitable powder base (carrier/diluent/excipient substance) such as mono-, di- or poly-saccharides (e.g., lactose or starch). Lactose is a commonly employed excipient for dry powder formulations. When a solid excipient such as lactose is employed, generally the particle size of the excipient will be much greater than the active ingredient to aid the dispersion of the formulation in the inhaler. Newer dry powder excipients are currently under investigation in the field and may provide optimal formulations for a dry powder fosfomycin/tobramycin combination product. Examples of such excipients include modified leucine including but not limited to tri-leucine and N-acetyl leucine. In one embodiment, the dry powder formulation comprises a blended composition comprising a fosfomycin component comprising micronized fosfomycin disodium and a tobramycin component comprising a spray-dried solution of pH-adjusted tobramycin base or tobramycin sulphate and N-acetyl leucine.

One example of a dry powder formulation is a dry powder formulation containing from about 1 to about 200 mg of fosfomycin and from about 0.1 to about 86 mg of tobramycin (wherein the ratio of components is as described above). In one embodiment, the formulation contains from about 10 to about 160 mg of fosfomycin and from about 2.5 to about 40 mg of tobramycin (wherein the ratio of components is as described above). In one particular preferred embodiment, the formulation contains from about 10 to about 160 mg of fosfomycin and from about 2.5 to about 40 mg of tobramycin wherein the ratio of fosfomycin to tobramycin is 4:1 (w/w of the pharmaceutical active form). In one particular preferred embodiment, the formulation contains from about 10 to about 40 mg of fosfomycin and from about 2.5 to about 10 mg of tobramycin wherein the ratio of fosfomycin to tobramycin is 4:1 (w/w of the pharmaceutical active form). In one embodiment, the dry powder formulation includes 10 mg fosfomycin and 2.5 mg tobramycin. In one embodiment, the dry powder formulation includes 20 mg fosfomycin and 5 mg tobramycin. In one embodiment, the formulation includes 40 mg fosfomycin and 10 mg tobramycin. In these formulation examples, both fosfomycin and tobramycin have a particle size suitable for inhalation (typically, from 1-5 microns). The formulation may also contain 25% (w/w of overall formulation mass) of a pharmaceutical grade excipient such as lactose monohydrate with a particle size from about 20 to about 300 μm.

In one embodiment, the composition is an inhalable pharmaceutical composition which is suitable for inhalation and delivery to the lung endobronchial space. Typically, such composition is in the form of an aerosol comprising particles for delivery using a nebulizer, pressurized metered dose inhaler (MDI), softmist inhaler, or dry powder inhaler (DPI). The aerosol formulation used in the methods of the present invention may be a liquid (e.g., solution) suitable for administration by a nebulizer, softmist inhaler, or MDI, or a dry powder suitable for administration by an MDI or DPI.

Aerosols used to administer medicaments to the respiratory tract are typically polydisperse, that is they are comprised of particles of many different sizes. The particle size distribution is typically described by the Mass Median Aerodynamic Diameter (MMAD) and the Geometric Standard Deviation (GSD). For optimum drug delivery to the endobronchial space the MMAD is in the range from about 1 to about 10 μm and preferably from about 1 to about 5 μm, and the GSD is less than 3, and preferably less than about 2. Aerosols having an MMAD above 10 μm are generally too large to reach the lungs when inhaled. Aerosols with a GSD greater than about 3 are not preferred for lung delivery as they deliver a high percentage of the medicament to the oral cavity. To achieve these particle sizes in powder formulations, the particles of the active ingredient may be size reduced using conventional techniques such as micronisation or spray drying. The desired fraction may be separated out by air classification or sieving. Preferably, the particles will be crystalline. For liquid formulations, the particle size is determined by the selection of a particular model of nebulizer, softmist inhaler, or MDI.

Aerosol particle size distributions are determined using devices well known in the art. For example a multi-stage Anderson cascade impactor or other suitable method such as those specifically cited within the US Pharmacopoeia Chapter 601 as characterizing devices for aerosols emitted from metered-dose and dry powder inhalers.

Non-limiting examples of dry powder inhalers include reservoir multi-dose inhalers, pre-metered multi-dose inhalers, capsule-based inhalers and single-dose disposable inhalers. A reservoir inhaler contains a large number of doses (e.g. 60) in one container. Prior to inhalation, the patient actuates the inhaler which causes the inhaler to meter one dose of medicament from the reservoir and prepare it for inhalation. Examples of reservoir DP's include but are not limited to the Turbohaler® by AstraZeneca and the ClickHaler® by Vectura.

In a pre-metered multi-dose inhaler, each individual dose has been manufactured in a separate container, and actuation of the inhaler prior to inhalation causes a new dose of drug to be released from its container and prepared for inhalation. Examples of multidose DPI inhalers include but are not limited to Diskus® by GSK, Gyrohaler® by Vectura, and Prahaler® by Valois. During inhalation, the inspiratory flow of the patient accelerates the powder out of the device and into the oral cavity. For a capsule inhaler, the formulation is in a capsule and stored outside the inhaler. The patient puts a capsule in the inhaler, actuates the inhaler (punctures the capsule), then inhales. Examples include the Rotohaler™ (GlaxoSmithKline), Spinhaler™ (Novartis), HandiHaler™ (IB), TurboSpin™ (PH&T). With single-dose disposable inhalers, the patient actuates the inhaler to prepare it for inhalation, inhales, then disposes of the inhaler and packaging. Examples include the Twincer™ (U Groningen), OneDose™ (GFE), Manta Inhaler™ (Manta Devices).

Generally, dry powder inhalers utilize turbulent flow characteristics of the powder path to cause the excipient-drug aggregates to disperse, and the particles of active ingredient are deposited in the lungs. However, certain dry powder inhalers utilize a cyclone dispersion chamber to product particles of the desired respirable size. In a cyclone dispersion chamber, the drug enters a coin shaped dispersion chamber tangentially so that the air path and drug move along the outer circular wall. As the drug formulation moves along this circular wall it bounces around and agglomerates are broken apart by impact forces. The air path spirals towards the center of the chamber exiting vertically. Particles that have small enough aerodynamic sizes can follow the air path and exit the chamber. In effect, the dispersion chamber works like a small jet mill. Depending on the specifics of the formulation, large lactose particles may be added to the formulation to aid in the dispersion through impact with the API particles.

The Twincer™ single-dose disposable inhaler appears to operate using a coin-shaped cyclone dispersion chamber referred to as an “air classifier.” See, U.S. Published Patent Application No. 2006/0237010 to Rijksuniversiteit Groningen. Papers published by the University of Groningen, have stated that a 60 mg dose of pure micronized colistin sulfomethate could be effectively delivered as an inhalable dry powder utilizing this technology.

In preferred embodiments, the aerosol formulation is delivered as a dry powder using a dry powder inhaler wherein the particles emitted from the inhaler have an MMAD in the range of about 1 μm to about 5 μm and a GSD about less than 2.

Examples of suitable dry powder inhalers and dry powder dispersion devices for use in the delivery of compositions according to the present invention include but are not limited to those disclosed in U.S. Pat. No. 7,520,278; U.S. Pat. No. 7,322,354; U.S. Pat. No. 7,246,617; U.S. Pat. No. 7,231,920; U.S. Pat. No. 7,219,665; U.S. Pat. No. 7,207,330; U.S. Pat. No. 6,880,555; U.S. Pat. No. 5,522,385; U.S. Pat. No. 6,845,772; U.S. Pat. No. 6,637,431; U.S. Pat. No. 6,329,034; U.S. Pat. No. 5,458,135; U.S. Pat. No. 4,805,811; and U.S. Published Patent Application No. 2006/0237010.

In one embodiment, the pharmaceutical formulation according to the invention is a dry powder for inhalation which is formulated for delivery by a Diskus®-type device. The Diskus® device comprises an elongate strip formed from a base sheet having a plurality of recesses spaced along its length and a lid sheet hermetically but peelably sealed thereto to define a plurality of containers, each container having therein an inhalable formulation containing a predetermined amount active ingredient either alone or in admixture with one or more carriers or excipients (e.g., lactose) and/or other therapeutically active agents. Preferably, the strip is sufficiently flexible to be wound into a roll. The lid sheet and base sheet will preferably have leading end portions which are not sealed to one another and at least one of the leading end portions is constructed to be attached to a winding means. Also, preferably the hermetic seal between the base and lid sheets extends over their whole width. To prepare the dose for inhalation, the lid sheet may preferably be peeled from the base sheet in a longitudinal direction from a first end of the base sheet.

In one embodiment, the pharmaceutical formulation according to the invention is a dry powder for inhalation which is formulated for delivery using a single-dose disposable inhaler, and particularly the Twincer™ inhaler. The Twincer™ inhaler comprises a foil laminate blister with one or more recesses and a lid sheet hermetically but peelably sealed thereto to define a plurality of containers. Each container has therein an inhalable formulation containing a predetermined amount of active ingredient(s) either alone or in admixture with one or more carriers or excipients (e.g., lactose). The lid sheet will preferably have a leading end portion which is constructed to project from the body of the inhaler. The patient would operate the device and thereby administer the aerosol formulation by 1) removing the outer packaging overwrap, 2) pulling the foil tab to uncover the drug in the blister and 3) inhaling the drug from the blister.

In another embodiment, a pharmaceutical composition according to the invention is delivered as a dry powder using a metered dose inhaler. Non-limiting examples of metered dose inhalers and devices include those disclosed in U.S. Pat. No. 5,261,538; U.S. Pat. No. 5,544,647; U.S. Pat. No. 5,622,163; U.S. Pat. No. 4,955,371; U.S. Pat. No. 3,565,070; U.S. Pat. No. 3,361,306 and U.S. Pat. No. 6,116,234 and U.S. Pat. No. 7,108,159. In a preferred embodiment, a compound of the invention is delivered as a dry powder using a metered dose inhaler wherein the emitted particles have an MMAD that is in the range of about 1 μm to about 5 μm and a GSD that is less than about 2.

The methods and uses according to the present invention may also be achieve using a liquid aerosol formulation suitable for delivery by inhalation. Liquid aerosol formulations for delivery to the lung or endobronchial space by inhalation may for example be formulated as aqueous solutions or suspensions or as aerosols delivered from pressurized packs, such as metered dose inhalers, with the use of suitable liquefied propellants, softmist inhalers, or nebulizers. Such aerosol compositions suitable for inhalation can be either a suspension or a solution and generally contain the active ingredient together with a pharmaceutically acceptable carrier or diluent (e.g., water, saline, or ethanol) and optionally one or more therapeutically active agents.

Aerosol formulations for delivery by pressurized metered dose inhalers typically further comprise a pharmaceutically acceptable propellant. Examples of such propellants include fluorocarbon or hydrogen-containing chlorofluorocarbon or mixtures thereof, particularly hydrofluoroalkanes, e.g., dichlorodifluoromethane, trichlorofluoromethane, dichlorotetrafluoroethane, especially 1,1,1,2-tetrafluoroethane, 1,1,1,2,3,3,3,-heptafluoro-n-propane or a mixture thereof. The aerosol composition may be excipient free or may optionally contain additional formulation excipients well known in the art such as surfactants e.g., oleic acid or lecithin and cosolvents e.g., ethanol. Pressurized formulations will generally be retained in a canister (e.g., an aluminum canister) closed with a valve (e.g., a metering valve) and fitted into an actuator provided with a mouthpiece.

In another embodiment, the aerosol formulation is delivered as a liquid using a metered dose inhaler. Non-limiting examples of metered dose inhalers and devices include those disclosed in U.S. Pat. No. 6,253,762, U.S. Pat. No. 6,413,497, U.S. Pat. No. 7,601,336, U.S. Pat. No. 7,481,995, U.S. Pat. No. 6,743,413, and U.S. Pat. No. 7,105,152.

In one particular embodiment, the aerosol formulation is delivered using a metered dose inhaler wherein the emitted particles have an MMAD that is in the range of about 1 μm to about 5 μm and a GSD that is less than about 2.

In one embodiment the aerosol formulation is suitable for aerosolization by a jet nebulizer, or ultrasonic nebulizer including static and vibrating porous plate nebulizers. Liquid aerosol formulations for nebulization may be generated by solubilizing or reconstituting a solid particle formulation or may be formulated with an aqueous vehicle with the addition of agents such as acid or alkali, buffer salts, and isotonicity adjusting agents. They may be sterilized by in process techniques such as filtration, or terminal processes such as heating in an autoclave or gamma irradiation. They may also be presented in non-sterile form.

In one embodiment, the fosfomycin plus tobramycin liquid aerosol formulation contains from about 1 to about 200 mg of fosfomycin and from about 0.1 to about 86 mg of tobramycin (wherein the ratio of components is as described above) per 1-5 mL of solution. In one embodiment, the formulation contains from about 10 to about 160 mg of fosfomycin and from about 2.5 to about 40 mg of tobramycin. In one particularly preferred embodiment, the formulation contains from about 10 to about 160 mg of fosfomycin and from about 2.5 to about 40 mg of tobramycin wherein the ratio of fosfomycin to tobramycin is 4:1 (w/w pharmaceutical active form). The solution is typically prepared using sterile water or sterile saline with a chloride concentration of at least 25 mM. In one embodiment, the liquid formulation for nebulization includes 10 mg fosfomycin and 2.5 mg tobramycin dissolved or suspended in 4 mL of solution. In one embodiment, the liquid formulation for nebulization includes 20 mg fosfomycin and 5 mg tobramycin dissolved or suspended in 4 mL of solution. In one embodiment, the liquid formulation for nebulization includes 40 mg fosfomycin and 10 mg tobramycin dissolved or suspended in 4 mL of solution. In one embodiment, the liquid formulation for nebulization includes 80 mg fosfomycin and 20 mg tobramycin dissolved or suspended in 4 mL of solution. In one embodiment, the liquid formulation includes 160 mg fosfomycin and 40 mg tobramycin dissolved or suspended in 4 mL of solution. In another embodiment, the liquid formulation includes 160 mg fosfomycin and 40 mg tobramycin dissolved or suspended in 2 mL of solution.

Patients can be sensitive to the pH, osmolality, and ionic content of a nebulized solution. Therefore these parameters should be adjusted to be compatible with fosfomycin plus tobramycin and tolerable to patients. The most preferred solution or suspension of fosfomycin plus tobramycin will contain a chloride concentration >30 mM at pH 4.5-8.0 and an osmolality of less than 1600 mOsm/kg, and preferably from about 800 to about 1000 mOsm/kg. The pH of the solution can be controlled by either titration with common acids (hydrochloric acid or sulfuric acid, for example) or bases (sodium hydroxide, for example) or via the use of buffers. Commonly used buffers include citrate buffers, acetate buffers, and phosphate buffers. Buffer strengths can range from 2 mM to 50 mM. The preferred pH range is 7-8 because the rate of hydrolysis of fosfomycin to the open-ring glycol impurity product (“fosfomycin Impurity A”) increases as fosfomycin is protenated; that is, as the solution becomes more acidic, fosfomycin rapidly degrades to fosfomycin impurity A, decreasing its potency.

Such formulations may be administered using commercially available nebulizers or other atomizer that can break the formulation into particles or droplets suitable for deposition in the respiratory tract. Non-limiting examples of nebulizers which may be employed for the aerosol delivery of a composition of the invention include pneumatic jet nebulizers, vented or breath enhanced jet nebulizers, or ultrasonic nebulizers including static or vibrating porous plate nebulizers.

A jet nebulizer utilizes a high velocity stream of air blasting up through a column of water to generate droplets. Particles unsuitable for inhalation impact on walls or aerodynamic baffles. A vented or breath enhanced nebulizer works in essentially the same way as a jet nebulizer except that inhaled air passes through the primary droplet generation area to increase the output rate of the nebulizer while the patient inhales.

In an ultrasonic nebulizer, vibration of a piezoelectric crystal creates surface instabilities in the drug reservoir that cause droplets to be formed. In porous plate nebulizers pressure fields generated by sonic energy force liquid through the mesh pores where it breaks into droplets by Rayleigh breakup. The sonic energy may be supplied by a vibrating horn or plate driven by a piezoelectric crystal, or by the mesh itself vibrating. Non-limiting examples of atomizers include any single or twin fluid atomizer or nozzle that produces droplets of an appropriate size. A single fluid atomizer works by forcing a liquid through one or more holes, where the jet of liquid breaks up into droplets. Twin fluid atomizers work by either forcing both a gas and liquid through one or more holes, or by impinging a jet of liquid against another jet of either liquid or gas.

The choice of nebulizer which aerosolizes the aerosol formulation is important in the administration of the active ingredient(s). Different nebulizers have differing efficiencies based their design and operation principle and are sensitive to the physical and chemical properties of the formulation. For example, two formulations with different surface tensions may have different particle size distributions. Additionally, formulation properties such as pH, osmolality, and permeant ion content can affect tolerability of the medication, so preferred embodiments conform to certain ranges of these properties.

In a particular embodiment, the formulation for nebulization is delivered to the endobronchial space as an aerosol having an MMAD between about 1 μm and about 5 μm and a GSD less than 2 using an appropriate nebulizer. To be optimally effective and to avoid upper respiratory and systemic side effects, the aerosol should not have a MMAD greater than about 5 μm and should not have a GSD greater than about 2. If an aerosol has an MMAD larger than about 5 μm or a GSD greater than about 2 a large percentage of the dose may be deposited in the upper airways decreasing the amount of drug delivered to the site of inflammation and bronchoconstriction in the lower respiratory tract. If the MMAD of the aerosol is smaller than about 1 μm then a large percentage of the particles may remain suspended in the inhaled air and may then be exhaled during expiration.

It should be understood that in addition to the ingredients particularly mentioned above, the formulations of this invention may include other agents conventional in the art having regard to the type of formulation in question.

### C. Dosing

The aerosol formulations may be presented in unit dosage form containing a predetermined amount of the active ingredients (fosfomycin and tobramycin) per unit dose, or in bulk form as for example in the case of compositions to be metered by an inhaler. Preferred unit dosage formulations for the aerosol formulation are those containing an effective amount of the combination of fosfomycin and tobramycin, or an appropriate fraction thereof.

The precise amount of fosfomycin and tobramycin contained in each unit dose may be optimized using conventional knowledge in the art based upon a number of factors, including the condition being treated, the route of administration, the bioavailability of the compounds, the species being treated, and the age, weight and condition of the patient. Unit dosage compositions may contain a monthly, weekly or daily dose or a sub-dose or an appropriate fraction thereof, of the active ingredients. Unit doses may be administered one or more times daily for the treatment of a particular condition.

Typically, the specific amounts of fosfomycin and tobramycin in the formulation, on a per dose basis will be in the range of from 1 to 200 mg of fosfomycin and from 0.1 to 86 mg of tobramycin (wherein the ratio of components is as described above). More particularly, the amount of fosfomycin and tobramycin in the formulation on a per dose basis will be in the range of from 10 to 160 mg of fosfomycin and from 2.5 to 40 mg of tobramycin. In one embodiment, the formulation, on a per dose basis, contains from about 20 to about 160 mg of fosfomycin and from about 5 to about 40 mg of tobramycin (wherein the ratio of fosfomycin to tobramycin is about 7 to about 9 parts by weight of fosfomycin and from about 1 to about 3 parts by weight of tobramycin). In one embodiment, the formulation, on a per dose basis, contains from about 20 to about 160 mg of fosfomycin and from about 5 to about 40 mg of tobramycin wherein the ratio of fosfomycin to tobramycin is about 4 to about 1 parts by weight of active pharmaceutical form. In one particular embodiment, the formulation, on a per dose basis, contains from about 10 to about 20 mg of fosfomycin and from about 2.5 to about 5 mg of tobramycin. In one particular embodiment, the formulation, on a per dose basis, contains about 10 mg of fosfomycin and about 2.5 mg of tobramycin. In one particular embodiment, the formulation, on a per dose basis, contains about 20 mg of fosfomycin and about 5 mg of tobramycin. In one particular embodiment, the formulation, on a per dose basis, contains about 40 mg of fosfomycin and about 10 mg of tobramycin. In one particular embodiment, the formulation, on a per dose basis, contains about 80 mg of fosfomycin and about 20 mg of tobramycin. In another particular embodiment, the formulation, on a per dose basis, contains about 160 mg of fosfomycin and about 40 mg of tobramycin.

The methods described herein are carried out by administering an effective amount of a combination of fosfomycin and tobramycin by inhalation, to the human with COPD. The term “effective amount”, as used herein, is an amount of the combination of fosfomycin and tobramycin which is sufficient in the subject to which it is administered, to elicit the biological or medical response of a cell culture, tissue, system, that is being sought, for instance by a researcher or clinician. In one embodiment, the effective amount is the amount needed to provide a desired level of drug in the secretions and tissues of the airways and lungs, or alternatively, in the bloodstream of a subject to be treated, to give an anticipated physiological response or desired biological effect when such a composition is administered by inhalation. For example an effective amount of the combination for reducing the frequency, severity or duration of an acute exacerbation of COPD in a human is an amount sufficient in the human to which it is administered to have the stated effect. In one embodiment an effective amount is an amount of the combination which is sufficient for the treatment of COPD in a human who is experiencing or at risk of experiencing an acute exacerbation of COPD. In another embodiment, the amount of the combination is an amount sufficient in the subject to which it is administered to reduce the frequency, severity or duration of an acute exacerbation of COPD in a human.

An effective amount of the combination of fosfomycin and tobramycin may contain less of each component than would be required for a therapeutic effect if each component were delivered separately. Thus, an effective amount of the combination of fosfomycin and tobramycin may contain a sub-therapeutic dose of one or both components. The precise effective amount of the combination will depend on a number of factors including but not limited to the species, age and weight of the subject being treated, the precise condition requiring treatment and its severity, the bioavailability, potency, and other properties of the compounds being administered, the nature of the formulation, the route of administration, and the delivery device, and will ultimately be at the discretion of the attendant clinician.

In one embodiment, an effective amount of the formulation contains from about 1 to about 200 mg of fosfomycin and from about 0.1 to about 86 mg of tobramycin (wherein the ratio of components is as described above). The selection of the specific dose for a patient will be determined by the attendant physician or clinician of ordinary skill in the art based upon a number of factors including those noted above. In one particular embodiment, the amount of fosfomycin and tobramycin in the formulation on a per dose basis will be in the range of from about 10 to about 160 mg of fosfomycin and from about 2.5 to about 40 mg of tobramycin (wherein the ratio of fosfomycin to tobramycin is about 7 to about 9 parts by weight of fosfomycin and from about 1 to about 3 parts by weight of tobramycin). In one embodiment, the formulation, on a per dose basis, contains from about 10 to about 160 mg of fosfomycin and from about 2.5 to about 40 mg of tobramycin wherein the ratio of fosfomycin to tobramycin is about 4:1 Fos:Tob by weight of the active pharmaceutical form. In one particular embodiment, the formulation, on a per dose basis, contains from about 10 to about 20 mg of fosfomycin and from about 2.5 to about 5 mg of tobramycin. In one particular embodiment, the formulation, on a per dose basis, contains about 10 mg of fosfomycin and about 2.5 mg of tobramycin. In one particular embodiment, the formulation, on a per dose basis, contains about 20 mg of fosfomycin and about 5 mg of tobramycin. In one particular embodiment, an effective amount of the formulation, on a per dose basis, contains about 40 mg of fosfomycin and about 10 mg of tobramycin. In one particular embodiment, an effective amount of the formulation, on a per dose basis, contains about 80 mg of fosfomycin and about 20 mg of tobramycin. In another particular embodiment, an effective amount of the formulation, on a per dose basis, contains about 160 mg of fosfomycin and about 40 mg of tobramycin.

Delivery of an effective amount of the combination of fosfomycin and tobramycin may entail delivery of a single dosage or multiple unit doses which may be delivered contemporaneously or separate in time over a designated period, such as 24 hours. Typically, the aerosol formulation will be administered four, three, or two times per day, or once per day (24 hours). In one embodiment, the aerosol formulation containing an effective amount of the combination will be administered two times per day (i.e., over a 24 hour period). In one particular embodiment, the aerosol formulation containing an effective amount of the combination will be administered twice per day (i.e., over a 24 hour period) for several consecutive days, particularly from 7 to 14 days, more particularly 7 days.

The aerosol formulation according to the present invention is designed for administration by inhalation. Inhaled antibiotics offer advantages over intravenous therapy because relatively high drug concentrations can be delivered to the site of infection with minimal systemic absorption, thus reducing the risk of side effects associated with IV exposure.

The lung dose of the aerosol formulation will vary depending upon the selected dose of each component drug in the aerosol formulation and the efficiency of the delivery device. It is well established that the efficiency of a nebulizer will vary from a dry powder inhaler and a metered dose inhaler. It is further well established that the efficiency may vary among different nebulizers, dry powder inhalers and metered dose inhalers. In one embodiment, a suitable lung dose of FTI for the methods and uses of the present invention will be about 10 mg fosfomycin and 2.5 mg tobramycin per dose.

### D. Preparation of Aerosol Formulations

The formulations may be prepared using conventional methods in the art of pharmacy. With the exception of bulk compositions such as those which may be employed in metered dose inhalers, the methods for preparing pharmaceutical compositions include the step of bringing the active ingredients into association with one or more carrier(s), diluent(s) and/or excipient(s) and optionally one or more accessory ingredients. In general the aerosol formulations are prepared by uniformly and intimately bringing into association the active ingredients with one or more liquid carriers, diluents or excipients or finely divided solid carriers, diluents or excipients, or both, and then, if necessary, appropriately modifying the product to obtain the desired particulate properties for inhalation.

In one embodiment, the present invention provides a process for the preparation of an aerosol formulation consisting of fosfomycin and tobramycin and optionally a pharmaceutically acceptable carrier, excipient or diluent, wherein the process comprises:

(a) preparing a particulate mixture of fosfomycin and tobramycin having a particle size suitable for inhalation (typically, from 1-5 microns); or
(b) admixing one or both of fosfomycin and tobramycin either individually or together with one or more pharmaceutically acceptable excipients, diluents and/or carriers; or
(c) solubilizing or suspending fosfomycin and tobramycin in a pharmaceutically acceptable solution.

### EXAMPLES

### Example 1

### Minimal Inhibitory Concentration (MIC) Studies

Three MIC Studies are reported below. The data below for two of the studies was previously published in PCT Publication No. WO2005/110022 to Gilead Sciences, Inc. (formerly Corus Pharma) and MacLeod, 2009 JAC.

### A. 9:1 Fos:Tob, 7:3 Fos:Tob and 5:5 Fos:Tob

The efficacy of antibiotics and antibiotic combinations against Gram-positive and Gram-negative bacteria representative of species that cause respiratory infections were evaluated in MIC assays. P. aeruginosa strains were isolated from lung sputum samples collected from cystic fibrosis patients, blood cultures, respiratory tract infections, and skin or soft tissue infections. H. influenzae, M. catarrhalis, and S. aureus, were isolated from respiratory tract infections. E. coli ATCC 25922, P. aeruginosa ATCC 27853, and S. aureus ATCC 29213 were used as quality control stains.

Method A: The MICs of fosfomycin alone, tobramycin alone, or combinations of fosfomycin plus tobramycin were determined by the agar-plate dilution method according to NCCLS guidelines (NCCLS, 2003). Bacterial strains were streaked onto Trypic Soy Agar plates (PML Microbiologicals, Wilsonville, Oreg.) containing 5% defibrinated sheep blood (hereafter referred to as blood agar plates) and incubated overnight at 35° C. Two to three bacterial colonies from the overnight cultures were inoculated into 3 mL of sterile normal saline, vortexed briefly, and adjusted to a 0.5 McFarland standard (NCCLS, 2003). The bacterial suspension was diluted 1:40 in sterile normal saline and served as the inoculum. Mueller-Hinton agar plates (hereafter referred to as MHA) were prepared by combining 16 g of agarose (Becton-Dickinson, Sparks, Md.), 22 g of Mueller-Hinton broth powder (Becton-Dickinson, Sparks, Md.), and adjusted to 1 L with distilled water. The agar was sterilized by autoclaving, cooled to 55° C., and supplemented with 25 μg/mL of glucose-6-phosphate (Sigma-Aldrich, St. Louis, Mo.). Twenty-five mL of cooled agar was aliquoted into 50 mL conical tubes and supplemented with appropriate concentrations of antibiotic to achieve concentrations ranging from 0.06 μg/mL to 512 μg/mL. After gently mixing the agar and antibiotic, the suspension was poured into sterile 100 mm petri dishes and allowed to solidify at room temperature. The antibiotic agar plates were inoculated with approximately 2×10⁴ CFU/spot with a 48-point inoculator (Sigma-Aldrich, St. Louis, Mo.). The MIC was defined as the lowest concentration of antibiotic (s) that prevented visible growth after incubation for 18-20 hours at 35° C. The activity of a particular antibiotic or antibiotic combination on large populations of P. aeruginosa was determined by calculating the MIC₅₀ and MIC₉₀ values. The MIC₅₀ value was defined as the concentration of antibiotic(s) which inhibited 50% of the P. aeruginosa strains. The MIC₉₀ value was defined as the concentration of antibiotic(s) which inhibited 90% of the P. aeruginosa strains (Wiedemann and Grimm, 1996).

Method B: The MICs of fosfomycin alone, tobramycin alone, or combinations of fosfomycin plus tobramycin were determined for P. aeruginosa strains in the presence of porcine gastric mucin to evaluate the effect of mucin on antibiotic activity. Methodologies were identical to that described in Method A above, with the exception that 2% (weight/volume) porcine gastric mucin (Sigma Chemical Co., St. Louis, Mo.) was added to the MHA prior to autoclaving.

Method C: The MICs of amikacin, arbekacin, dibekacin, gentamicin, kanamycin, netilimicin, neomycin, streptomycin, and tobramycin alone were determined for P. aeruginosa ATCC 27853 by the broth-microdilution method according to NCCLS standards (NCCLS, 2003). E. coli ATCC 25922 and S. aureus ATCC 29213 were used as quality control stains. Bacterial strains were streaked onto blood agar plates and incubated at 35° C. for 18 hours. Two to three bacterial colonies from the overnight cultures were inoculated into 3 mL of sterile normal saline, vortexed briefly, and adjusted to a 0.5 McFarland standard (NCCLS, 2003). The bacterial suspension was diluted 1:100 in cation-adjusted Mueller-Hinton broth (hereafter referred to as CAMHB). Fifty microliters of bacterial inoculum (approximately 2×10⁵ CFU/mL) was pipeted into individual wells of 96-well plates containing 50 μl of CAMHB (Remel, Lenexa, Kanas) supplemented with 2-fold dilutions of antibiotics ranging in concentration from 0.125 μg/mL to 128 μg/mL. The MIC was defined as the lowest concentration of antibiotic (s) that prevented visible growth after incubation at 35° C. for 18-24 hours.

The in vitro potency of fosfomycin and tobramycin alone and in combination against a panel of Gram-negative and Gram-positive bacteria representative of species that cause respiratory tract infections is shown in Table 1.

TABLE 1
MIC values of fosfomycin and tobramycin alone and in
combination against Gram-negative and Gram-positive bacteria.
MIC (μg/mL)

| | | | | | |
|---|---|---|---|---|---|
| | | | 9:1 | 7:3 | 5:5 |
| Bacteria | Fosfomycin | Tobramycin | Fos:Tob | Fos:Tob | Fos:Tob |

P. aeruginosa

| | | | | | |
|---|---|---|---|---|---|
| COR-003 | 16 | 0.5 | 8 | 4 | 1 |
| COR-009 | 1024 | 2048 | 1024 | 512 | 2048 |
| COR-021 | 8 | 64 | 8 | 8 | 8 |
| COR-027 | 128 | 0.5 | 8 | 2 | 1 |

H. influenzae

| | | | | | |
|---|---|---|---|---|---|
| COR-042 | 8 | 0.5 | 4 | 2 | 1 |
| COR-049 | 128 | 0.5 | 4 | 1 | 1 |

M. catarrhalis

| | | | | | |
|---|---|---|---|---|---|
| COR-109 | 4 | 8 | 4 | ND | ND |
| COR-113 | 4 | 8 | 4 | ND | ND |

S. aureus

| | | | | | |
|---|---|---|---|---|---|
| COR-051 | 1 | 0.25 | 2 | 1 | 0.5 |
| (MSSA) | | | | | |
| COR-055 | 4 | 0.25 | 4 | 1 | 0.5 |
| (MRSA) | | | | | |
| COR-059 | 2 | 128 | 2 | 4 | 4 |
| COR-060 | 2 | 64 | 1 | 4 | 8 |
| (GISA) | | | | | |

S. pneumoniae

| | | | | | |
|---|---|---|---|---|---|
| COR-061 | 32 | 64 | 16 | 32 | 32 |
| COR-068 | 8 | 16 | 16 | 16 | 16 |

ND = Not determined.

The data show that the fosfomycin:tobramycin combinations have antibacterial activity against a broad spectrum of Gram-positive and Gram-negative bacteria including those which are important in the pathogenesis of COPD.

Table 2 shows the MIC₅₀, and MIC₉₀ values of fosfomycin and tobramycin alone and in combination for 100 P. aeruginosa strains isolated from lung sputum samples from cystic fibrosis patients.

TABLE 2

| | MIC₅₀ (μg/mL) | | MIC₉₀ (μg/mL) | |
|---|---|---|---|---|
| Antibiotic | Mucin (−) | Mucin (+) | Mucin (−) | Mucin (+) |
| Fosfomycin | 64 | 64 | =512 | 128 |
| Tobramycin | 2 | 16 | 16 | 64 |
| 9:1 Fos:Tob | 16 | 32 | 64 | 128 |
| 4:1 Fos:Tob | 8 | 32 | 64 | 128 |
| 7:3 Fos:Tob | 8 | 16 | 64 | 64 |

This study demonstrated that in the absence of mucin, tobramycin was the most active antibiotic. Combining fosfomycin and tobramycin resulted in a significant decrease in the MIC₅₀ and MIC₉₀ values compared to fosfomycin alone. In the mucin binding model (+mucin), the fosfomycin:tobramycin combinations had MIC₅₀ and MIC₉₀ values to comparable tobramycin alone.

The foregoing data is a subset of data published in PCT Publication no. WO2005/110022 to Gilead Sciences Inc. (formerly Corus Pharma).

### B. Published MIC Studies of 4:1 Fos:Tob

In a previously published study, susceptibility testing was conducted to assess the antimicrobial activity of FTI in comparison with the individual antibiotic components, fosfomycin and tobramycin, against P. aeruginosa and other Gram-positive and Gram-negative bacteria. MacLeod, 2009 JAC. MICs were determined by agar plate dilution and broth microdilution methods according to the NCCLS Methods for Dilution Antimicrobial Susceptibility Tests for Bacterial That Grow Aerobically, 6^{th} Ed. Approved Standard M7-A6, 2003 and NCCLS Performance Standards for Antimicrobial Susceptibility Testing, 14^{th} Ed. Approved Standard M100-S13 2003. (Activity was also evaluated using a modified susceptibility assay that incorporates 2% mucin to model the effects of sputum binding on antibacterial activity. Time-kill curves for antibiotic activity in mucin are reported below.) The MIC was defined as the lowest concentration of antibiotic that prevented visible growth after incubation at 35° C. for 18-24 hr. FTI MIC values were expressed as the concentration of both drugs (example, FTI MIC of 8 mg/L=6.4 mg/L fosfomycin+1.6 mg/L tobramycin). Vancomycin and ciprofloxacin MICs were determined only for the S. aureus isolates.

Table 3 summarizes the MICs at which 50% (MICA and 90% (MIC₉₀) of the clinical isolates were inhibited by FTI, fosfomycin or tobramycin.

TABLE 3
MIC values of fosfomycin and tobramycin alone and in combination against Gram-negative and Gram-positive bacteria.

| | No. | | | | | | | | | |
|---|---|---|---|---|---|---|---|---|---|---|
| | of | MIC (mg/L) | | | | | | | | |
| | Isol. | FTI | | | Tobramycin | | | Fosfomycin | | |
| Organism | tested | range | MIC₅₀ | MIC₉₀ | range | MIC₅₀ | MIC₉₀ | range | MIC₅₀ | MIC₉₀ |
| S. aureus | 16 | 0.5-16 | 2 | 8 | 0.125-512 | 0.5 | 256 | 0.125-16 | 2 | 4 |
| S. | 8 | 4-32 | ND | ND | 16-64 | ND | ND | 8-32 | ND | ND |
| pneumoniae | | | | | | | | | | |
| S. | 5 | 16-32 | ND | ND | 16-64 | ND | ND | 16-64 | ND | ND |
| pyogenes^{b} | | | | | | | | | | |
| E. faecalis | 5 | 32 | ND | ND | 8-512 | ND | ND | 32 | ND | ND |
| E. coli | 22 | 0.125-1 | 0.5 | 1 | 0.5-1 | 1 | 1 | 0.25-4 | 0.5 | 2 |
| H. | 16 | ≦0.13-4 | 0.5 | 2 | 0.5-1 | 1 | 1 | 0.25-4 | 0.5 | 2 |
| Influenzae | | | | | | | | | | |
| Klebsiella | 22 | 0.5-16 | 1 | 8 | 0.13-<512 | 0.13 | 16 | 0.5-16 | 4 | 16 |
| spp.^{b} | | | | | | | | | | |
| M. catarrhalis | 5 | 0.5-1 | ND | ND | 0.5-1 | ND | ND | 4-16 | ND | ND |
| P. | 60 | 1-256 | 4 | 128 | 0.13->512 | 1 | 128 | 1->512 | 32 | 128 |
| aeruginosa | | | | | | | | | | |
| (non-CF) | | | | | | | | | | |
| P. | 100 | 1-128 | 8 | 64 | 0.25->512 | 2 | 16 | 4->512 | 64 | 512 |
| aeruginosa | | | | | | | | | | |
| (CF) | | | | | | | | | | |
| S. | 17 | 8-256 | 64 | 128 | 2->512 | 64 | 256 | 32-512 | 64 | 128 |
| maltophilia | | | | | | | | | | |
| B. cepacia | 20 | .05->512 | 512 | >512 | 1->512 | 64 | 512 | 512->512 | >512 | >512 |
| complex | | | | | | | | | | |

^{b}MICs were determined by the broth microdilution method. All other MICs were determined by the agar dilution method.
ND = not determined due to the small number of isolates examined. MacLeod, 2009 JAC.

MacLeod, 2009 JAC reports the following:
FTI had high activity against the 16 random S. aureus strains, and moderate activity against S. pneumoniae, S. pyogenes, and E. faecalis. Twelve of the 16 S. aureus strains were categorized as MRSA. The FTI MIC₅₀ value (2 mg/L) was nearly identical to that of vancomycin (1 mg/L) and was superior to that of ciprofloxacin (>4 mg/L) for S. aureus. FTI was also active against single linezolid-resistant (CO₅₉) and glycopeptides-intermediate S. aureus (GISA) (C060) isolates, with MICs of 2 and 1 mg/L respectively.
Among the Gram-negative organisms examined FTI has the lowest MIC₅₀ for E. coli (0.5 mg/L), H. influenzae (0.5 mg/L), Klebsiella spp. (1 mg/L) and P. aeruginosa (non-CF, 4 mg/L; and CF, 8 mg/L) strains. FTI also has high activity against M. catarrhalis strains, but poor activity against S. maltophilia and B. cepacia complex. Against tobramycin-resistant and high fosfomycin MIC (=128 mg/L) strains, FTI had MICs comparable to that of the most active single antibiotic component. Tobramycin had the lowest MIC₅₀ and MIC₉₅ values for the CF (2 and 16 mg/L) and non-CF P. aeruginosa (1 and 128 mg/L) strains. Fosfomycin has potent activity against S. aureus, H. influenzae, E. coli and Klebsiella spp. It showed moderate activity against P. aeruginosa and S. maltophilia and poor activity against B. cepacia complex
   C. Minimal Inhibitory Concentration (MIC) Studies with 4:1 Fos:Tob

In vitro susceptibility data was generated against 1332 recent clinical isolates selected to represent a range of species likely or potentially involved in COPD. The studies were carried out according to the standard methods of the Clinical Laboratory Standards Institute (CLSI) by the Clinical Microbiology Institute (CMI) {9725 SW Commerce Circle, Wilsonville, Oreg. 97070}.

Broth microdilution and agar dilution tests were performed according to the current version of the NCCLS/CLSI document M7-A7 at the time the studies were conducted. MIC trays were produced at CMI using cation adjusted Mueller-Hinton broth (CAMHB, DIFCO lot #7306781). The medium was supplemented with lysed horse blood (Hemostat lot #H0318) for testing the streptococci or made up as Haemophilus Test Medium (HTM) for testing H. influenzae. All media containing fosfomycin or FTI were supplemented with 25 μg/ml of glucose-6-phosphate. The CLSI standards specify that “The approved MIC susceptibility testing method is agar dilution. Broth dilution should not be performed.” This is due to a generally poor correlation between agar dilution and broth microdilution methods. Although broth microdilution was performed in this study, all comparisons of FTI to fosfomycin or tobramycin were made on the basis of the agar dilution results.

Agar dilution plates were prepared using BBL dehydrated Mueller-Hinton agar media (lot #8134155) supplemented as needed with 5% sheep blood (Hema Resources lot #0911-100140-04) or made up as HTM agar. As with the broth media, all agar dilution media containing fosfomycin or FTI were supplemented with 25 μg/ml of glucose-6-phosphate.

FTI was tested at a fixed fosfomycin to tobramycin ratio of 4:1 and was tested over a range of 256/64 μg/ml down to 0.12/0.03 μg/ml. Fosfomycin alone was tested over a range of 256 μg/ml to 0.12 μg/ml. Tobramycin alone was tested over a range of 32 μg/ml down to 0.015 μg/ml.

Broth microdilution test panels and agar dilution plates were prepared at CMI. Fosfomycin powder (lot #077K1668), tobramycin (lot #068K1232), oxacillin (lot #018K0610) penicillin (lot #095K0625) and ampicillin (lot #106K0689) were purchased from Sigma. The oxacillin, penicillin, and ampicillin were used for the phenotypic classification of S. aureus, S. pneumoniae, and H. influenzae, respectively.

In order to determine if there are differences between agar dilution and microbroth dilution techniques in this study, all strains were tested in parallel by both methods. The results were analyzed using MIC/MIC regression plots and bar graphs showing broth microdilution MICs minus agar dilution MICs.

The following quality control strains were tested daily as testing progressed: E. coli ATCC 25922, S. aureus ATCC 29213, E. faecalis ATCC 29212, P. aeruginosa ATCC 27853, S. pneumoniae ATCC 49619, H. influenzae ATCC 49247, H. influenzae ATCC 10211. Additional in-house quality control strains were used at the time of tray production in order to assure “on-scale” quality control throughout the range of concentrations tested.

The antimicrobial activity of FTI against bacterial agents associated with COPD is summarized in Table 3. This table demonstrates the MIC₅₀ and MIC₉₀. FTI shows good in vitro activity against all of the bacterial species found in lungs of patients with COPD.

TABLE 4
MIC values of fosfomycin and tobramycin alone and in combination
against Gram-negative and Gram-positive bacteria.

| | Number | | | | | | |
|---|---|---|---|---|---|---|---|
| | of | FTI | | Tobramycin | | Fosfomycin | |
| | isolates | MIC (μg/mL) | | MIC (μg/mL) | | MIC (μg/mL) | |
| Bacteria | tested | MIC₅₀ | MIC₉₀ | MIC₅₀ | MIC₉₀ | MIC₅₀ | MIC₉₀ |
| H. influenzae β- | 62 | 0.25 | 1 | 4 | 8 | 0.25 | 8 |
| lactamase neg | | | | | | | |
| H. influenzae β- | 39 | 0.25 | 1 | 4 | 4 | 0.5 | 2 |
| lactamase pos | | | | | | | |
| S. pneumoniae | 39 | 8 | 16 | 32 | >32 | 16 | 16 |
| penicillin | | | | | | | |
| suscept. | | | | | | | |
| S. pneumoniae | 32 | 8 | 32 | 32 | >32 | 16 | 32 |
| Penicillin | | | | | | | |
| resistant | | | | | | | |
| M. catarrhalis | 103 | 1 | 1 | 0.12 | 0.25 | 8 | 16 |
| Enterobacter | 603 | 1 | 4 | 0.5 | 8 | 2 | 32 |
| spp | | | | | | | |
| S. aureus | 104 | 1 | 2 | 0.25 | 0.5 | 4 | 4 |
| (MSSA) | | | | | | | |
| S. aureus | 103 | 2 | 8 | 0.5 | >32 | 4 | 8 |
| (MRSA) | | | | | | | |

Data presented represents the MIC collected using the agar dilution method and not the microbroth method. When FTI broth microdilution MICs were compared to those of agar dilution MICs, in general there was >95% agreement between the two methods. The percent agreement between fosfomycin broth microdilution and agar dilution was even less at 74.6% (data not presented).

### Example 2

### Minimal Bactericidal Concentration (MBC)/MIC

### A. MBC/MIC Values of 9:1 Fos:Tob, 4:1 Fos:Tob and 7:3 Fos:Tob

The MBCs of fosfomycin and tobramycin alone for P. aeruginosa ATCC 27853, E. coli ATCC 25922, and S. aureus ATCC 29213 were determined by the broth microdilution method according to NCCLS standards (NCCLS, 1999). This data was previously published in PCT Publication No. WO20051110022 to Gilead Sciences Inc. (formerly Carus Pharma). Bacterial strains were streaked onto blood agar plates and incubated at 35° C. for 18-24 hours. Two to three bacterial colonies from the overnight cultures were inoculated into 3 mL of sterile normal saline, vortexed briefly, and adjusted to a 0.5 McFarland standard (NCCLS, 2003). Fifty microliters of bacterial inoculum (approximately 2×10⁵ CFU/mL) was pipeted into individual wells of 96-well plates containing 50 μl of CAMHB (Remel, Lenexa, Kanas) supplemented with 2-fold dilutions of antibiotics ranging in concentration from 0.125 μg/mL to 128 μg/mL. Plates were incubated at 35° C. for 18-24 hours and MIC determined as described in Example 1, Method C. The contents of wells showing no growth (MIC and above) were mixed with a pipetor and duplicate 10 μl samples spread onto blood agar plates. Culture plates were incubated at 35° C. for 18-24 hours and the number of bacterial colonies on each plate enumerated manually. Rejection values were determined by NCCLS methods which considers the final inoculum size, single or double sampling, pipetting error, and the Poisson distributuion of sample responses (NCCLS, 1999). For example, if the final inoculum was 5×10⁵ CFU/mL and duplicate samples were evaluated, the lowest dilution having fewer than a total of 25 colonies was considered the MBC. The MBC was defined as the =3 Log₁₀ decrease in CFU/mL of the original inoculum as described by NCCLS standards (NCCLS, 1999). The MBC/MIC ratios were calculated by dividing the MBC by the MIC.

Table 5 shows the MBC/MIC values of fosfomycin and tobramycin alone and 9:1, 4:1, and 7:3 combinations for P. aeruginosa ATCC 27853, E. coli ATCC 25922, and S. aureus ATCC 29213. For P. aeruginosa, the MBC/MIC values of the 9:1, 4:1, and 7:3 combinations were identical to tobramycin alone. This finding was not observed with E. coli or S. aureus.

TABLE 5
MBC/MIC values of fosfomycin and tobramycin alone and 9:1,
4:1, and 7:3 combinations of fosfomycin and tobramycin.

| | MBC/MIC | | |
|---|---|---|---|
| | P. aeruginosa | E. coli | S. aureus |
| Antibiotic | ATCC 27853 | ATCC 25922 | ATCC 29213 |
| Fosfomycin | =8 | 8 | 1 |
| Tobramycin | 1 | 1 | 8 |
| 9:1 Fos:Tob | 1 | 1 | 2 |
| 4:1 Fos:Tob | 1 | 2 | 4 |
| 7:3 Fos:Tob | 1 | 2 | 4 |

### B. MBC/MIC Ranges of Clinical Strains Against 4:1 Fos:Tob

MacLeod, 2009 JAC also reports the MBC of FTI compared to tobramycin and fosfomycin. MBCs were determined according to CLSI (formerly NCCLS) guidelines. The MBC was defined by a =3 Log₁₀ decrease in cfu/mL of the original inoculum. The MBC/MIC ratio was calculated by dividing the MBC (mg/L) by the MIC (mg/L). Results are reported in Table 6.

TABLE 6

| | Range of MBC/MIC | | | |
|---|---|---|---|---|
| | No. of | | | |
| Organism | strains | FTI | tobramycin | fosfomycin |
| S. aureus | 10 | 1-2 | 1-2 | 1-8 |
| S. pneumoniae | 7 | 1-4 | 1-2 | 1-16 |
| P. aeruginosa (non-CF) | 10 | 1-4 | 1-2 | 2-8 |
| P. aeruginosa (CF) | 8 | 1-4 | 1-4 | 2->16 |
| E. coli | 10 | 1-4 | 1-4 | 1-8 |
| Klebsiella spp. | 5 | 1 | 1-4 | 1-4 |
| H. Influenzae | 6 | 1-8 | 1-2 | 1-8 |

### Id.

MacLeod, 2009 JAC reports the following:
FTI and tobramycin were bactericidal against the S. aureus (100%), S. pneumoniae (100%), P. aeruginosa (100%), E. coli (100%), Klebsiella spp. (100%) and H. influenzae (83% and 100%, respectively) strains. Fosfomycin was bactericidal against S. aureus (80%), S. pneumoniae (86%), P. aeruginosa (78%), E. coli (90%), Klebsiella spp. (100%) and H. influenzae (83%) strains. FTI and tobramycin had MBC/MIC ratios=8, suggesting that both antibiotics work by killing bacteria rather than by inhibiting bacterial growth.

### Example 3

### Time Kill Studies of Fti Relative to Fosfomycin and Tobramycin Alone

### A. Time-Kill Curves for 9:1 Fos:Tob, 4:1 Fos:Tob and 7:3 Fos:Tob

Time-kill experiments were performed in the presence of 2% porcine gastric mucin to evaluate the effect of mucin and protein binding on antibiotic activity. These studies were previously reported in PCT Publication no. WO2005/110022 to Gilead Sciences, Inc. (formerly Carus Pharma). Two to three bacterial colonies were inoculated into 10 mL CAMHB and incubated at 35° C. in a shaking water bath (250 rpm) for 18-24 hours. A 1:40 dilution of the overnight culture was made in 10 mL of fresh CAMHB and incubated at 35° C. in a shaking water bath (250 rpm) for 1-2 hours. The resulting culture was adjusted to a 0.5 McFarland standard (NCCLS, 2003). To reduce variability in the bacterial inoculum size when comparing multiple antibiotics, one master tube of CAMHB containing 2% (weight/volume) of porcine gastric mucin was inoculated with a 1:200 dilution of bacterial inoculum (approximately 5×10⁵ CFU/mL), supplemented with 25 μg/mL of glucose-6-phosphate, and briefly vortexed. Ten milliliter aliquots were then pipeted into 50 mL conical tubes. Fosfomycin alone, tobramycin alone, and combinations of fosfomycin plus tobramycin were added to the culture medium at concentrations equal to 1, 2, 4, and 8-fold multiples of the fosfofomycin MIC (4 μg/mL) for P. aeruginosa ATCC 27853. The killing activity of fosfomycin plus tobramycin was also compared to the killing activity of the individual components. For example, 16 μg/mL of a 9:1 fosfomycin:tobramycin combination was compared to killing activity of 12.8 μg/mL of fosfomycin alone and 3.2 μg/mL of tobramycin alone. A no drug control was conducted in each experiment. Cultures were incubated with antibiotic at 35° C. in a shaking (250 rpm) water bath for 24 hours. Bacterial killing was determined at 0, 1, 2, 4, 6, and 24 hours by making 10-fold serial dilutions of the cultures in sterile normal saline, and spreading 100 μl aliquots on blood agar plates. Culture plates were incubated at 35° C. for 18-24 hours and the number of colonies enumerated manually. The limit of detection for the colony counting method was 1 Log₁₀. Time-kill curves were constructed by plotting the Log₁₀ number of CFU/mL culture versus time. Antibiotic concentrations that reduced the original inoculum by =3 Log₁₀ CFU/mL were considered bactericidal, and concentrations that reduced the original inoculum by =2 Log₁₀ CFU/mL were defined as bacteriostatic (NCCLS, 1999). Synergism was defined as a reduction of bacterial colony counts of =2 Log₁₀, CFU/mL with the antibiotic combinations compared to the most active single antibiotic (NCCLS, 1999).

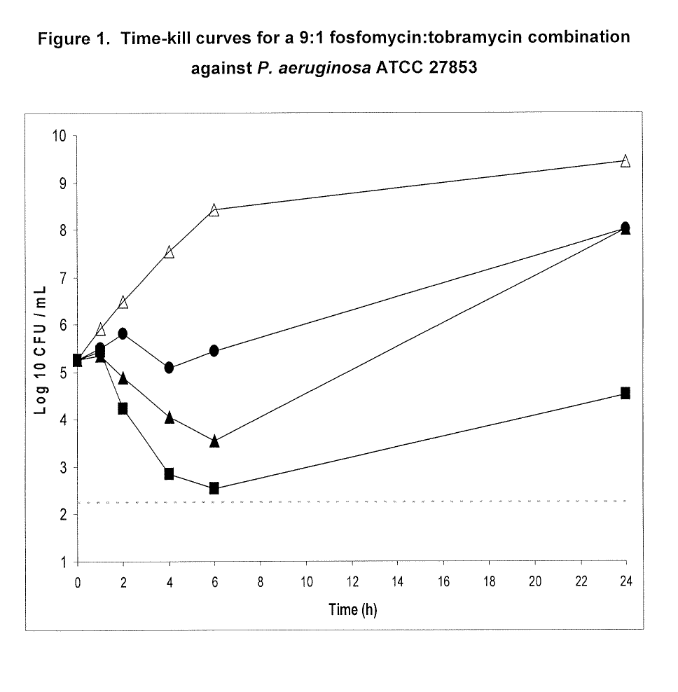

FIG. 1 shows the time-kill curves for a 9:1 fosfomycin:tobramycin combination and demonstrate rapid bactericidal killing of P. aeruginosa ATCC 27853 compared to bacteriostatic killing of fosfomycin and tobramyicn alone.

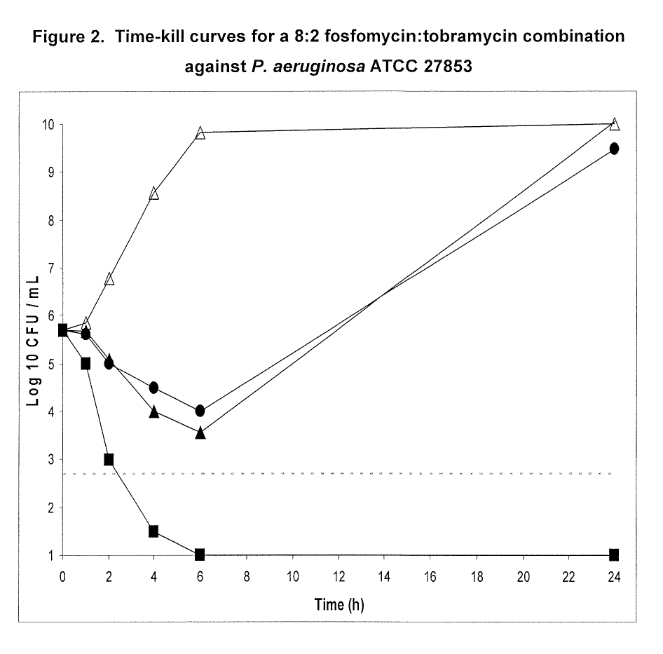

FIG. 2 shows time-kill curves for 4:1 fosfomycin:tobramycin combinations and demonstrate rapid bactericidal killing of P. aeruginosa ATCC 27853. At 4×MIC, FTI (12.8 μg/mL fosfomycin+3.2 μg/mL tobramycin) was rapidly bactericidal against P. aeruginosa, and demonstrated superior activity relative to the individual component concentrations of fosfomycin (12.8 μg/mL) or tobramycin (3.2 μg/mL), which exhibited bacteriostatic activity at these concentrations (FIG. 2). At 24 hours, FTI remained bactericidal against P. aeruginosa ATCC 27853 (blood culture isolate routinely used in labs), while re-growth was observed during exposure to either fosfomycin or tobramycin alone.

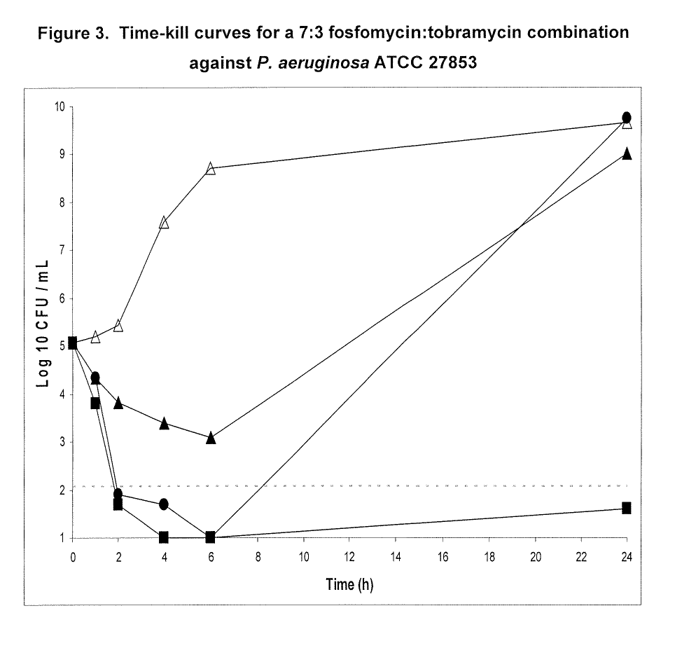

FIG. 3 shows time-kill curves for 7:3 fosfomycin:tobramycin combinations and demonstrate rapid bactericidal killing of P. aeruginosa ATCC 27853. At concentrations of 16 μg/mL, the killing activity of the combination was superior to fosfomycin and tobramycin alone.

B. Evaluation of Concentration Versus Time-Dependent Killing of P. aeuriginosa

Dose-response time-kill studies were conducted to further evaluate the antimicrobial activity of each individual component, as well as the FTI combination, against P. aeuriginosa. In addition to providing pertinent information regarding whether each antibiotic is bactericidal or bacteriostatic and exerts time-dependent or concentration-dependent activity, these studies permitted a more thorough comparison of the effective concentrations of FTI in relation to fosfomycin or tobramycin alone.

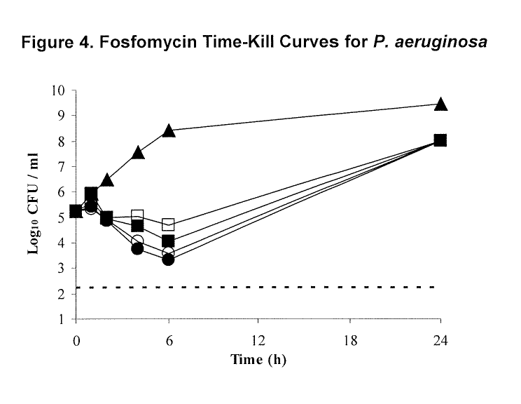

In all instances, the killing activity was evaluated in the presence of 2% mucin. In FIG. 4 the time-kill curve for fosfomycin alone is presented. Fosfomycin exhibited time-dependent killing and was bacteriostatic for P. aeuriginosa; increasing the fosfomycin concentration did not produce a significant increase in the rate or degree of bacterial killing (FIG. 4).

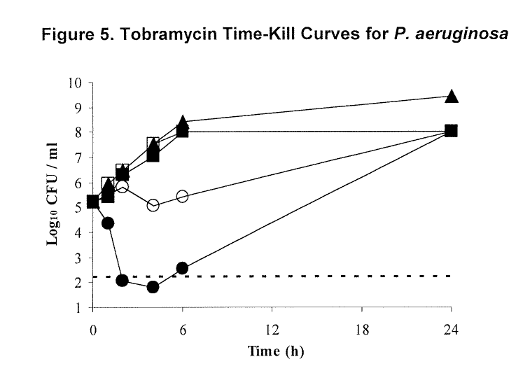

The time-kill curve for tobramycin alone is presented in FIG. 5. Tobramycin exhibited concentration-dependent killing and was rapidly bactericidal for P. aeuriginosa, but re-growth occurred within 24 hours (FIG. 5). Increasing the tobramycin concentration significantly increased both the rate and extent of killing.

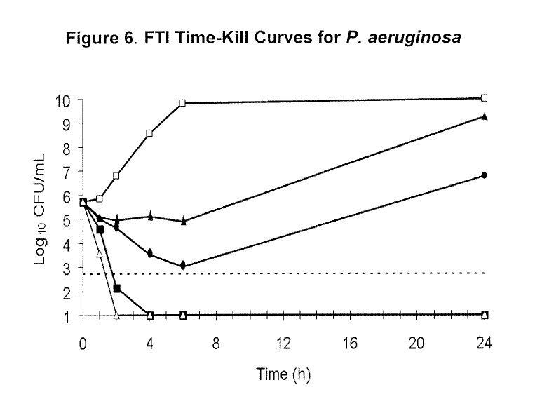

The time-kill curve for 4:1 Fos:Tob is presented in FIG. 6. Similar to tobramycin, FTI exhibited concentration-dependent killing and was rapidly bactericidal for P. aeuriginosa (FIG. 6). Unlike tobramycin, no re-growth was observed within 24 hours at bactericidal concentrations of FTI. The fact that both FTI and tobramycin exhibit concentration-dependent killing suggests that the primary mechanism of action may be inhibition of protein synthesis.

### Example 4

### Effect on Protein and Cell Wall Synthesis and Drug Uptake

It is currently believe that the primary mechanism of action of FTI appears to be fosfomycin mediated enhanced uptake of tobramycin leading to inhibition of protein synthesis.

Protein and cell wall peptidoglycan biosynthesis were determined by measuring the incorporation of tritiated (³H) amino acids (³H-aa) (GE Healthcare Bio-Sciences Corp.; Picataway, N. J.) and N-acetyl-D-Glucosamine (³H-NAG) (GE Healthcare Bio-Sciences), respectively. An overnight culture of P. aeruginosa ATCC 27853 was diluted 1:1000 in 50 mL CAMHB+2% mucin in a 125 mL Erlenmeyer flask and incubated at 37° C., 200 rpm for 1.5 h. Two milliliters of early log phase cultures (˜2×10⁷ CFU/mL) were pulsed with 10 μCi of ³H-aa (1.93 GBq/milliatom carbon) or 10 μCi of ³H-NAG (296 GBq/mmol) for 1 h at 37° C., 200 rpm. FTI, fosfomycin, or tobramycin were then added to cultures and incubated as described above for up to an additional 4 h. At various time points, 100 μl aliquots (triplicate) of culture were removed and macromolecules precipitated with 10% TCA (VWR). Samples were harvested onto glass fiber filters (GFC) (PerkinElmer; Waltham, Mass.), washed two times with 35 mL of normal saline to remove unincorporated isotope followed by one wash with 35 mL of 90% ETOH (VWR). Counts per minute (CPM) were determined using a Wallac MicroBeta Trilux (PerkinElmer).

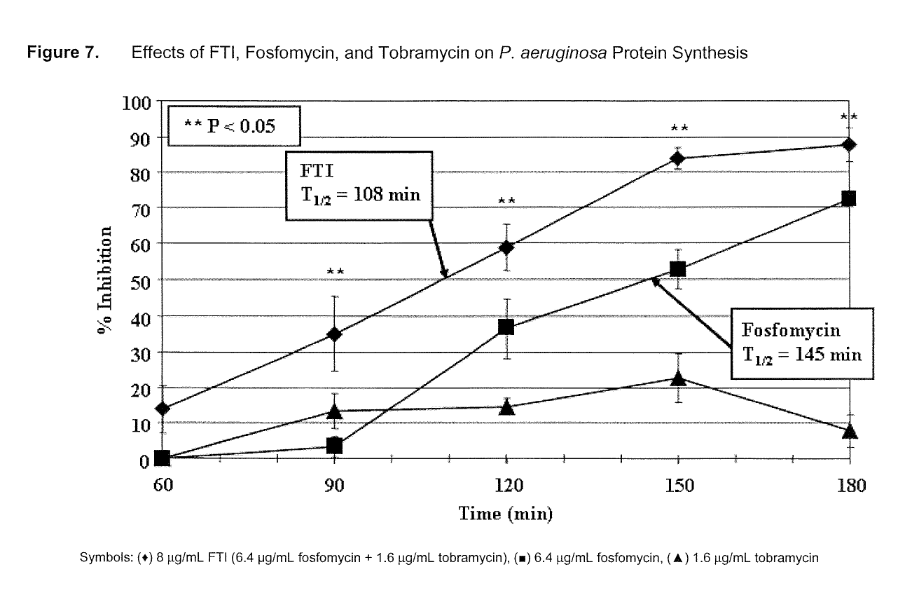

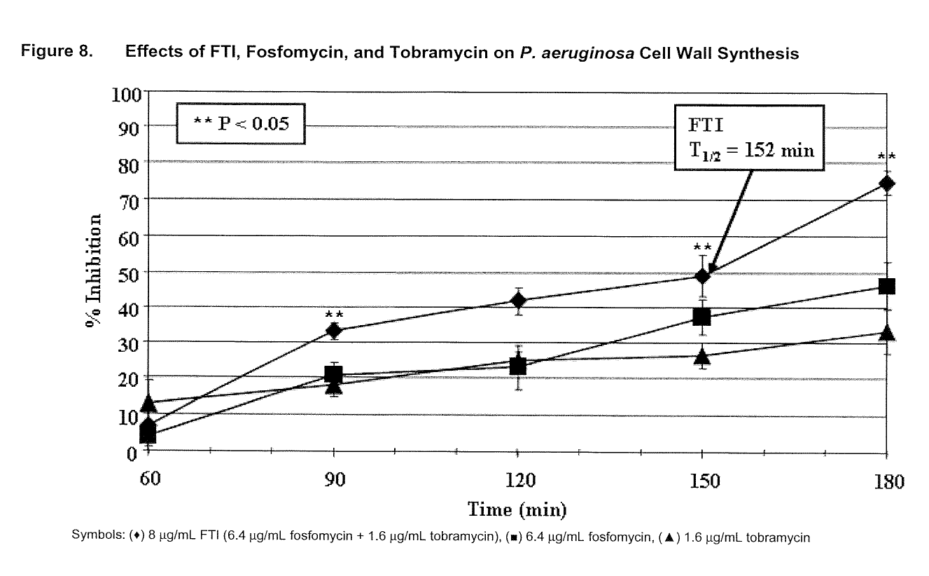

After 60 and 90 minutes of drug exposure, fosfomycin (6.4 μg/mL) and tobramycin (1.6 μg/mL) demonstrate poor protein synthesis inhibition in P. aeruginosa relative to FTI (6.4-1.6 μg/mL) (FIG. 7). Drug uptake studies, presented below, demonstrate enhanced uptake of tobramycin in the presence of fosfomycin, suggesting that the enhanced protein synthesis inhibition of FTI at 60 and 90 minutes is caused by tobramycin mediated mechanisms.

The time-response studies presented in FIGS. 7 and 8 suggest that FTI acts initially through inhibition of protein synthesis via the tobramycin mode of action. FTI (6.4-1.6 μg/mL) rapidly inhibited 50% (T_{1/2}) of protein synthesis by 108 minutes compared to 6.4 μg/mL fosfomycin (T_{1/2}=145 min) and 1.6 μg/mL tobramycin (T_{1/2} not determined [>180 min]). In contrast, FTI (6.4-1.6 μg/mL) caused a more gradual inhibition of cell wall biosynthesis (T_{1/2}=152 min), while neither fosfomycin (6.4 μg/mL) nor tobramycin (1.6 μg/mL) reached 50% inhibition within 180 min.

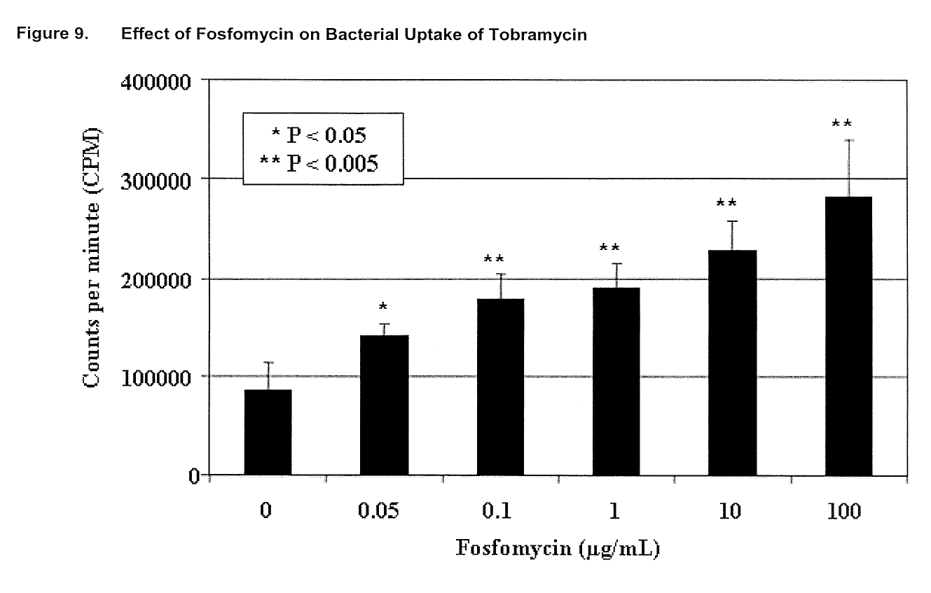

Antibiotic uptake was determined by measuring incorporation of ³H-tobramycin (540 mCi/mmol, Moravek Biochemicals; Brea, Calif.). An overnight culture of P. aeruginosa ATCC 27853 was diluted in nutrient broth (NB) (Difco & BBL; Sparks, Md.) to an OD₆₂₅ of 0.013 and incubated at 37° C. with shaking (250 rpm) until it reached an OD₆₂₅ of ˜0.5. Cells were harvested by centrifugation (6000×g, room temperature, 5 min), washed once in NB and resuspended in pre-warmed NB to an OD₆₂₅ of 0.25. Fosfomycin was added 0, 0.05, 0.1, 1, 10, and 100 mg/L and the cultures incubated for 3 min at 37° C. with shaking (250 rpm). ³H-tobramycin (2.3 mg/L) was added to each tube and the cultures were incubated at 37° C. with shaking (250 rpm) for an additional 2 min. Five milliliter volumes were filtered through 0.45 μm nitrocellulose membrane filters (Whatman Inc., Florham Park, N.J.). Uptake of [³H]-tobramycin was measured in the presence of increasing concentrations of fosfomycin (FIG. 9). The addition of 10 μg/mL fosfomycin resulted in a 170 percent increase in (³H)-tobramycin uptake relative to the no-fosfomycin control. The molecular mechanism responsible for fosfomycin mediated enhanced uptake of tobramycin is unknown. The macromolecular biosynthesis and drug uptake studies support the hypothesis that the enhanced ability of FTI to inhibit P. aeruginosa protein synthesis may be due to fosfomycin-mediated induction of tobramycin uptake into bacterial cells.

### Example 5

### Single-Step Mutation Frequency Studies

A. Single-step Mutation Frequency for 4:1 Fos:Tob Compared to Fosfomycin and Tobramycin Alone Against S. aureus and P. aeruginosa

Development of resistance after a single exposure to antibiotic was determined using four clinical and one reference strain of S. aureus (ATCC 29213) and P. aeruginosa (ATCC 27853). Late log-phase cultures (10⁹-10¹⁰ cfu) were spread onto Mueller-Hinton agar (BBL, Sparks, Md. USA) plates containing 4×, 8× and 16× the MIC of each antibiotic. The culture plates were incubated at 35° C. for 48 hr and the number of colonies on each plate was enumerated manually. The frequency of resistance was calculated by dividing the number of bacteria growing at the defined antibiotic concentration by the number of bacterial in the inoculum. See, J L. Martinez, et al., 2000 Antimicrob Agent Chemother 44:1771-1777. MIC values were calculated for representative spontaneous mutants and compared with those for the parental strain. Table 7 shows the frequencies of spontaneous single-step mutations leading to antibiotic resistance.

TABLE 7
Spontaneous Mutation Frequencies Resulting in Development
of Antibiotic Resistance for P. aeruginosa ATCC
27853 and S. aureus ATCC 29213

| Organism and | MIC | Mutation Frequency | | |
|---|---|---|---|---|
| Antibiotic | (μg/mL) | 4X MIC | 8X MIC | 16X MIC |
| P. aeruginosa | | | | |
| FTI | 4 | 4.6 × 10⁻⁷ | 5.1 × 10⁻⁷ | 4.1 × 10⁻⁸ |
| Tobramycin | 0.5 | 3.0 × 10⁻⁵ | 2.2 × 10⁻⁷ | 1.6 × 10⁻⁸ |
| Fosfomycin | 4 | 7.2 × 10⁻⁴ | 1.1 × 10⁻⁵ | 7.2 × 10⁻⁵ |
| S. aureus | | | | |
| FTI | 2 | <3.1 × 10⁻¹⁰ | <3.1 × 10⁻¹⁰ | <1.8 × 10⁻¹⁰ |
| Tobramycin | 0.25 | 1.6 × 10⁻⁷ | 3.9 × 10⁻⁸ | <1.8 × 10⁻¹⁰ |
| Fosfomycin | 2 | 2.6 × 10⁻⁸ | 5.0 × 10⁻⁸ | <1.8 × 10⁻¹⁰ |

Spontaneous mutation frequency for FTI is lower than fosfomycin and tobramycin.

Development of resistance after a single exposure to antibiotic was lowest for FTI followed by tobramycin and fosfomycin (Table 5). Against P. aeruginosa FTI

(4×MIC) was superior to tobramycin by two orders of magnitude. Against S. aureus (MSSA) FTI (4×MIC) had a mutation frequency three orders of magnitude less than tobramycin and two orders less than fosfomycin.

B. Single-Step P. aeruginosa Mutation Frequencies±Mucin

The airways of COPD patients contain sputum, a thick viscous secretion containing elevated levels of mucin, DNA from inflammatory cells, filamentous actin (F-actin), lipids and peptides. The altered biophysical properties of sputum impair mucocilliary clearance resulting in airway obstructions and predispose the patients to bacterial infections. Furthermore, microscopic analysis of sputum from CF patients demonstrates that P. aeruginosa grows in aggregations or microcolonies and exhibit increased resistance to antibiotics due to production of biofilms. Whole sputum and individual components of sputum, including mucin, DNA and F-actin bundles have also been shown to decrease the activity of cationic antibiotics and peptides through electrostatic binding. Once bound to mucin, antibiotics are unable to exert their pharmacological effects. This contributes to the development of bacterial resistance. In vitro models that incorporate the properties of airway sputum could provide additional insights for developing antibiotics for chronic respiratory infections.

Development of resistance after a single exposure to antibiotic was determined using four clinical and one reference strain of S. aureus and P. aeruginosa. Late log-phase cultures (10⁹-10¹⁰ cfu) were spread onto Mueller-Hinton agar (BBL, Sparks, Md. USA) plates±2% mucin (2 g mucin/100 mL media) containing 4×, 8×, 16× and 32× the MIC of each antibiotic. The culture plates were incubated at 35° C. for 48 hr and the number of colonies on each plate was enumerated manually. The frequency of resistance was calculated by dividing the number of bacteria growing at the defined antibiotic concentration by the number of bacterial in the inoculum. See, J L Martinez, et al., 2000 Antimicrob Agent Chemother 44:1771-1777. MIC values were calculated for representative spontaneous mutants and compared with those for the parental strain.

TABLE 8
P. aeruginosa Mutation Frequencies ± Mucin

| | | | Test Article | | |
|---|---|---|---|---|---|
| | | MIC | 4:1 Fos:Tob | Fos | Tob |
| | | (μg/mL) | 4 | 4 | 0.5 |
| 4x MIC | Mucin | − | 3 × 10⁻⁹ | 3 × 10⁻⁵ | 4 × 10⁻⁷ |
| | | + | 5 × 10⁻⁶ | 7 × 10⁻⁶ | no effect |
| 8x MIC | Mucin | − | 5 × 10⁻⁹ | 8 × 10⁻⁶ | 9 × 10⁻⁸ |
| | | + | 6 × 10⁻⁷ | 3 × 10⁻⁶ | no effect |
| 16x MIC | Mucin | − | 5 × 10⁻⁹ | 3 × 10⁻⁶ | >5 × 10⁻⁹ |
| | | + | 5 × 10⁻⁹ | 3 × 10⁻⁶ | 2 × 10⁻⁵ |
| 32x MIC | Mucin | − | 5 × 10⁻⁹ | 3 × 10⁻⁶ | >5 × 10⁻⁹ |
| | | + | 5 × 10⁻⁹ | 2 × 10⁻⁶ | 2 × 10⁻⁶ |

The data demonstrate that in the presence of mucin, a major component of normal airway mucus and COPD sputum, antibiotic resistance frequencies for 4:1 Fos:Tob were 25,000 and 1,700 times lower than for tobramycin and fosfomycin alone.

### Example 6

### Effects of FTI on Bacterial Virulence Factors Associated with Inflammatory Response

Bacterial virulence factors are bacterial products or mechanisms that cause damage to host tissues (e.g. adhesions, toxins, proteases). Bacteria important in the COPD produce numerous virulence factors that are critical for establishing chronic infections of the airways and initiation of immune and inflammatory responses that cause lung damage and loss of lung function. B. Henderson, et al., “Bacterial Modulins: a Novel Class of Virulence Factors Which Cause Host Tissue Pathology by Inducing Cytokine Synthesis” 1996 Microbiol. Rev. 60(2):316-341; A. Clatworthy, et al., “Targeting virulence: a new paradigm for antimicrobial therapy” 2007 Nature Chem Bio 3(9):541-548; and C. Caldwell, et al., “Pseudomonas aeruginosa Exotoxiin Pyocyanin Cases Cystic Fibrosis Airway Pathogenesis” 5 Nov. 2009 Am J. Pathol (ahead of print). Therapies that inhibit production of such virulence factors would reduce the ability of bacteria to infect the airways, reduce the inflammatory response in the airways and slow destruction of lung function.

### DNA Microarray Analysis

Exposure of P. aeruginosa to Subinhibitory Concentrations of Antibiotics.

Time-kill experiments were performed according to a modified CLSI method. Antibiotics were evaluated alone and in combination at multiples of the MIC in cation-adjusted Mueller Hinton Broth (CAMHB) (Remel; Lenexa, Kans., USA) containing 20 μL PGM. Bacterial cultures and FTI (Fos:Tob 4:1; 12.8 μg/mL Fosfomycin and 3.2 μg/mL tobramycin) were incubated at 37° C. in a shaking water bath (200 rpm) and viability assessed by the plate count method at 0, 1, 2, 4, 6 and 24 h. A no drug control was run in each assay. Total bacterial RNA was isolated from approximately 10⁷ CFU's of P. aeruginosa ATCC 27853 with an RNAeasy kit (Qiagen, USA) after a 1 h exposure to FTI. RNA was treated with RNAse free DNAase to remove contaminating genomic DNA. RNA was further purified with kit. RNA concentration and purity was determined spectrophotometrically (Ratio of 260 nm/280 nm).

### RNA Labeling/Hybridization and Scanning/Image Analysis

cDNA synthesis, labeling, hybridization and microarray analysis were performed at the Center for Expression Arrays (CEA), Department of Microbiology, University of Washington School of Medicine, Seattle, Wash. 98195. The detection calls present, absent, or marginal for each transcript were determined using default parameters. For the FTI treatment group, a comparison expression analysis was conducted with Affymetrix LCOS v1.3, using the untreated control sample as baseline. Absolute signal intensities for transcript were normalized by globally scaling all probe sets to the default target signal intensity of 500.

TABLE 9
P. aeruginosa virulence genes down-regulated by FTI (16 μg/mL).

| | | | Log 2 |
|---|---|---|---|
| PAO-1 | Gene | Gene | expression |
| ORF | name | description | change |
| PA0024 | | hemolysin | −1 |
| PA0040 | | hemagglutinin | −1.6 |
| PA0045 | — | curli | −2.2 |
| PA0046 | — | curli | −2.5 |
| PA0047 | — | curli | −2.9 |
| PA0408 | pilG | twitching motility | −1.6 |
| PA0410 | pilI | twitching motility | −1.2 |
| PA0411 | pilJ | twitching motility | −1.1 |
| PA1077 | flgB | flagellar basal-body rod protein | −1.2 |
| PA1078 | flgC | flagellar basal-body rod protein | −1.6 |
| PA1079 | flgD | flagellar basal-body rod modification | −1.1 |
| PA1080 | flgE | flagellar hook protein | −1.1 |
| PA1081 | flgF | flagellar basal body rod protein | −1 |
| PA1082 | flgG | flagellar basal body rod protein | −1.4 |
| PA1083 | flgH | flagellar L-ring protein precursor | −1.1 |
| PA1097 | fleQ | transcriptional regulator | −1.3 |
| PA1098 | fleS | two-component sensor | −1.2 |
| PA1099 | fleR | two-component response regulator | −1.6 |
| PA1100 | fliE | flagellar hook basal body complex protein | −1.5 |
| PA1101 | fliF | flagellar M-ring protein | −1.4 |
| PA1718 | pscE | type III export protein | −1.2 |
| PA1719 | pscF | type III export protein | −1.2 |
| PA1720 | pscG | type III export protein | −1 |
| PA2652 | — | chemotaxis | −1.4 |
| PA2653 | — | chemotaxis | −1.4 |
| PA2654 | — | chemotaxis | −1.3 |
| PA2867 | — | chemotaxis | −2.9 |
| PA2868 | — | chemotaxis | −1.8 |
| PA5040 | pilQ | fimbiae | −1.9 |
| PA5041 | pilP | fimbiae | −2.1 |
| PA5042 | pilO | fimbiae | −2.3 |
| PA5043 | pilN | fimbiae | −2.1 |
| PA5044 | pilM | fimbiae | −1.7 |

The data demonstrate that FTI inhibits expression of numerous bacterial virulence factors known to be associated with production of inflammatory response.

### Example 7

### In Vivo Studies—Rat Bacterial Pneumonia Model

The following experiment and results are also reported in MacLeod, 2009 JAC.

Animals were handled according to the guidelines for the Care and use of Laboratory Animals. See, NRC (National Research Council) Guide for the Care and Use of Laboratory Animals. Washington, D.C., USA: National Academy Press 1996. All animal protocols were approved by an IRB/Ethics Committee. Male Sprague-Dawley rats (180-200 g) were obtained from Charles River Laboratories (Hollister, Calif., USA) and acclimatized for 5 days prior to use. Animals were housed individually in ventilated cages, fed Purina Lab Diet ad libitum and allowed free access to water.

Antibiotic efficacy was determined using a rat bacterial pneumonia model. See, H A Cash, et al., 1979 Am Rev Respir Dis 119:453-459. Rats were anaesthetized with isoflurane, and ˜10³ cfu of P. aeruginosa C177 in 2% agar solution were instilled into the lungs with an oral gavage needle. The inoculum was deposited at the first bifurcation and distributed throughout the lungs by inspiration. Animals were allowed to recover for 18 hr post-infection. Each experiment consisted of pre-treatment (n=5-7), saline control with isoflurane, and 100 μL of antibiotic solution or saline was instilled in the trachea using a Microsprayer™ (Penn-Century In., Philadelphia, Pa., USA). Antibiotics were administered intratracheally twice daily for 3 days.

Animals were euthanized by intraperitoneal administration of sodium pentobarbital. The pre-treatment control group was harvested 18 hr post-infection, and the saline and treatment groups 18 hr after the last antibiotic exposure. Lungs were removed aseptically, homogenized in sterile normal saline and viable bacteria determined by the colony count method. Statistical differences between the saline control group and treatment groups were evaluated by the Mann-Whitney Rank Sum Test using GraphPad Prism® software package version 3.03 (GraphPad Software, Inc., San Diego, Calif., USA).

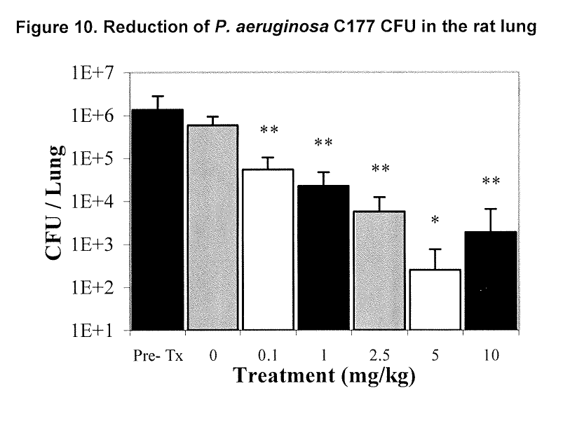

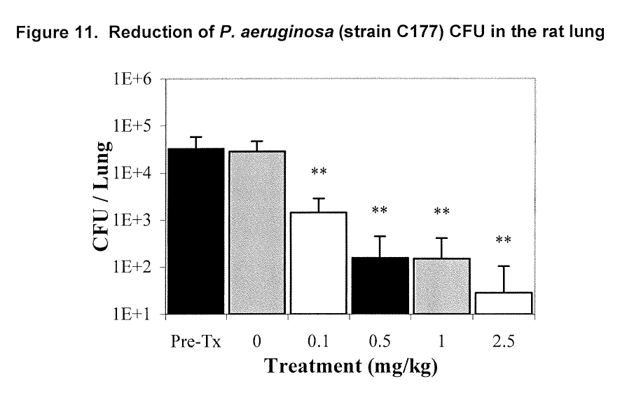

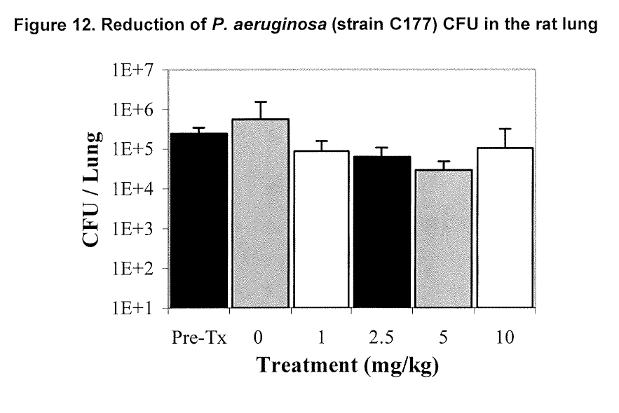

The reduction of P. aeruginosa Strain C177 CFU in the rat lung after intratracheal administration of 0.1, 1, 2.5, 5 and 10 mg/kg of 4:1 Fos:Tob is reported in FIG. 10. The reduction of P. aeruginosa Strain C177 CFU in the rat lung after intratracheal administration of 0.1, 0.5, 1, and 2.5 mg/kg of tobramycin is reported in FIG. 11. The reduction of P. aeruginosa Strain C177 CFU in the rat lung after intratracheal administration of 1, 2.5, 5 and 10 mg/kg of fosfomycin is reported in FIG. 12.

In the absence of antibiotic treatment, cfu/lung decreased <1 Log₁₀ at days 4 and 7 post-infection. Intratracheal administration of FTI showed progressively greater killing of P. aeruginosa with increasing dose (FIG. 10). In subsequent experiments, complete eradication of the C177 infection was seen with 5 and 12.5 mg/kg 4:1 Fos:Tob. Tobramycin showed 3-Log₁₀ bacterial killing at 2.5 mg/kg (FIG. 11). Administration of tobramycin doses higher than 3 mg/kg resulted in complete eradication of P. aeruginosa infection, while doses=0.5 mg/kg did not result in bacterial killing. A reduction in cfu/lung was not observed after administration of =10 mg/kg fosfomycin (FIG. 12).

A 5 mg/kg dose of 4:1 Fos:Tob (FIG. 10) contains only 1 mg/kg of tobramycin, yet demonstrates greater killing than achieved by 1 mg/kg of tobramycin alone (FIG. 11). In the rat model, an ^{˜}500 fold reduction in lung burden was achieved with 1 mg/kg of tobramycin while an ^{˜}1400 fold reduction lung burden was achieved with 4:1 Fos:Tob containing 1 mg/kg of tobramycin. Notably, the initial lung burden in the 4:1 Fos:Tob study was 10 times greater than the lung burden in the tobramycin study. These data support the enhanced levels of 4:1 Fos:Tob killing activity observed in mucin time-kill experiments (FIG. 6).

### Example 8

### Sputum Drug Concentrations of FTI

In order to assess the amount of drug delivered to the respiratory track, drug concentration was measured in expectorated sputum for patients with CF or bronchiectasis. Pharmacokinetic data for sputum from both doses and patient type is presented in Table 7. The fosfomycin and tobramycin range for patient type and dose was 1751-2974 μg/g and 326-881 μg/g, respectively.

TABLE 10
Sputum drug concentrations following aerosolization of FTI

| Cₘₐₓ (μg/g) | |
|---|---|
| Fosfomycin | Tobramycin |

| | 80:20 | 160:40 | 80:20 | 160:40 |
|---|---|---|---|---|
| Patients with: | mg FTI | mg FTI | mg FTI | mg FTI |
| Cystic Fibrosis | 2073 | 2974 | 580 | 881 |
| Bronchiectasis | 1751 | 1981 | 326 | 587 |

The data demonstrate that drug concentration levels of FTI achieved by aerosolization are extremely high and sufficient to kill the bacterial pathogens important in COPD.

### Example 9

### Study Design for Evaluating Safety and Efficacy in Humans

The FDA Guidance: Acute Bacterial Exacerbations of Chronic Bronchitis in Patients with Chronic Obstructive Pulmonary Disease: Developing Antimicrobial Drugs for Treatment specifically addresses the development of antimicrobial drugs for the treatment of exacerbations in this indication. The clinical program for the prevention of acute exacerbations in COPD patients will consider any applicable clinical development requirements from the guidance, taking into consideration the guidance's objective of treatment over prevention of exacerbations.

Clinical studies in COPD patients with a history of recurrent acute exacerbations will focus on reducing the frequency, duration or severity of exacerbations and also evaluate changes in FEV₁, Quality of Life, and health care utilization. The clinical development path will follow trial designs similar to other studies currently being conducted for reduction of acute exacerbations.

The proposed Phase 2 study is a multicenter, randomized, double-blind, placebo-controlled study to evaluate the safety and efficacy of FTI in patients with moderate-to-severe COPD (i.e., baseline FEV₁=70% predicted) who are >40 years old and have had a minimum of 2 acute exacerbations in the preceding 12 months. The Phase 2 study will include two FTI arms−1) Fos:Tob 40 mg:10 mg and 2) Fos:Tob 20 mg:5 mg; a tobramycin 10 mg arm and matching placebo. In all arms, drug will be administered by DPI twice a day for 7 days of every 28-day period, for a total of at least 6 months. Each arm of the study is expected to require about 150 patients.

The primary endpoint will be time to first acute exacerbation requiring treatment, with onset of acute exacerbation determined by the clinical investigator using a protocol-specified definition. The FDA draft guidance on COPD states that the definition should be “clinically meaningful” and include criteria such as worsening of dyspnea, increased sputum volume, increased purulence of sputum, worsening in symptoms requiring changes in treatment, or worsening of symptoms requiring urgent treatment or hospitalization. Key secondary endpoints will include number, severity and duration of acute exacerbation as assess by both type of treatment (e.g. oral antibiotics, hospitalization) and clinical assessment; time to second exacerbation; and change from baseline in FEV₁% predicted. The FDA Guidance noted above recommends the use of a patient reported outcome (PRO) primary endpoint. One potential PRO instrument, the Exacerbations from Pulmonary Disease Tool (EXACT-PRO), is currently being developed for evaluating clinical response in acute bacterial exacerbations of chronic bronchitis in patients with COPD. In addition to change from baseline in FEV₁% predicted, other safety endpoints will also be assessed, including change of MICs from screening to end of study.

Based upon the outcome of the Phase 2 study, the more efficacious dose of FTI (assuming comparable safety) would then be evaluated in two, 12-month Phase 3 studies, which would also include a tobramycin arm at the dose of tobramycin component of FTI in the Phase 3 trial, and a matching placebo arm. The primary endpoint of these studies would be number, severity and duration of acute exacerbations. The study would ideally be powered to demonstrate superior efficacy of FTI compared with tobramycin.

### Example 10

### Fosfomycin/Tobramycin Aerosol Formulations for Dry Powder Inhalation

### A. 4:1 Fosfomycin/Tobramycin Inhalation Powder

Fosfomycin disodium (8.4346 g, 6.3175 g free acid) having a particle size suitable for inhalation (typically, from 1-5 microns) is added to the mixing container and tumbled by hand. 94.2% tobramycin base (8.3587 g) having a particle size suitable for inhalation (typically, from 1-5 microns) and fosfomycin disodium (16.7260 g, 15.7559 g free acid) are added to the mixing container. The mixing container is then placed into the Turbula® shaker-mixer on a setting of 22 rpm for 5 minutes. Additional fosfomycin disodium (16.1600 g, 15.227 g free acid) is added to the mixing container and then placed into the Turbula® shaker-mixer on a setting of 22 rpm for 15 minutes. The fosfomycin/tobramycin ratio was calculated to be 4:1.

B. 4:1 Fosfomycin/Tobramycin with 25% (w/w) Lactose Inhalation Powder

Lactose monohydrate (10.6250 g) was added to the mixing container and tumbled by hand. 94.2% tobramycin base (5.3125 g) having a particle size suitable for inhalation (typically, from 1-5 microns) is added to the mixing container. The mixing container is then placed into the Turbula® shaker-mixer on a setting of 22 rpm for 10 minutes. Fosfomycin disodium (26.5625 g, 25.0219 g free acid) having a particle size suitable for inhalation (typically, from 1-5 microns) is added to the mixing container and then placed into the Turbula® shaker-mixer on a setting of 22 rpm for 15 minutes. The fosfomycin/tobramycin ratio was calculated to be 4:1.

### Example 11

### Fosfomycin/Tobramycin Aerosol Formulations for Nebulization

### A. 9:1 Fosfomycin/Tobramycin Solution

Fosfomycin disodium (18.057 g, 13.99 g free base) is dissolved in 250 mL of water. To the resulting solution is added 1.56 g of 97.5% tobramycin base. The pH of the solution is adjusted to approximately 7.6 by the addition of 3.98 mL of 4.5 N HCl. The solution is diluted to a total volume of 500 mL with water and filtered through a 0.2 μm Nalge Nunc 167-0020 membrane filter. The final pH will be approximately 7.8, the osmolality will be approximately 540 mOsmol/kg, the fosfomycin/tobramycin ratio is calculated to be 9:1, and the chloride concentration will be approximately 36 mM.

### B. 4:1 Fosfomycin/Tobramycin Solution

A solution of fosfomycin/tobramycin in a 8:2 ratio was prepared. 3.1680 g of fosfomycin disodium (2.4013 g free base) was dissolved in 50 ml water. 0.6154 g of 97.5% tobramycin base (0.6000 g of pure tobramycin base) was dissolved in the fosfomycin solution. The pH was adjusted by adding 0.910 mL of 6 M HCl. The solution was diluted to 100 mL with water. The final pH of the solution was 7.65, the osmolality was 477 mOsmol/kg, and the chloride concentration was 54.6 mM. The final fosfomycin/tobramycin ratio was calculated to be 4:1.

### C. 7:3 Fosfomycin/Tobramycin Solution

Using the procedure described for the 9:1 solution, above, a solution of fosfomycin/tobramycin in a 7:3 ratio was prepared; 17.466 g of fosfomycin disodium (13.239 g free base) is dissolved in water, 5.819 g of 97.5% tobramycin base (5.674 g of pure tobramycin base) is added to the solution, and the pH of the combined solution is adjusted by adding 10.66 mL of 4.5 N HCl. The final pH of the solution will be approximately 7.7, the osmolality will be approximately 560 mOsmol/kg, the fosfomycin/tobramycin ratio will be 7:3, and the chloride concentration will be approximately 96 mM.

That which is claimed is:

1. A method for treating a human with chronic obstructive pulmonary disease (COPD) who is experiencing or at risk of experiencing acute exacerbation of COPD, comprising administering by inhalation to said human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

2. The method according to claim 1 wherein the acute exacerbation of COPD is manifested by one or more symptoms selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, and said method comprises reducing the frequency, severity or duration of one or more of said symptoms.

3. The method according to claim 1, wherein the acute exacerbation is acute exacerbation of chronic bronchitis in a human with COPD.

4. The method according to claim 1, wherein the acute exacerbation is acute bacterial exacerbation of chronic bronchitis in a human with COPD.

5. The method according to claim 1 wherein said formulation comprises about 4 parts by weight of fosfomycin to about 1 parts by weight of tobramycin.

6. The method according to claim 1, wherein said formulation is in a pharmaceutically acceptable solution and is suitable for administration by a nebulizer.

7. The method according to claim 1, wherein said formulation is a dry powder suitable for administration by a dry powder inhaler or metered dose inhaler.

8. The method according to claim 1, wherein said formulation consists of from about 1 to about 200 mg of fosfomycin and from about 0.1 to about 86 mg of tobramycin.

9. A method for reducing the frequency, severity or duration of acute exacerbation of COPD in a human, comprising administering by inhalation to said human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

10. The method according to claim 9 wherein the acute exacerbation of COPD is manifested by one or more symptoms selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, and said method comprises reducing the frequency, severity or duration of one or more of said symptoms.

11. The method according to claim 9, wherein the acute exacerbation is acute exacerbation of chronic bronchitis in a human with COPD.

12. The method according to claim 9, wherein the acute exacerbation is acute bacterial exacerbation of chronic bronchitis in a human with COPD.

13. The method according to claim 9 wherein said formulation comprises about 4 parts by weight of fosfomycin to about 1 parts by weight of tobramycin.

14. The method according to claim 9, wherein said formulation is in a pharmaceutically acceptable solution and is suitable for administration by a nebulizer.

15. The method according to claim 9, wherein said formulation is a dry powder suitable for administration by a dry powder inhaler or metered dose inhaler.

16. The method according to claim 9, wherein said formulation consists of from about 1 to about 200 mg of fosfomycin and from about 0.1 to about 86 mg of tobramycin.

17. A method for treating one or more symptoms of acute exacerbation of COPD in a human, comprising administering by inhalation to said human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

18. The method according to claim 17, wherein said one or more symptoms are selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion.

19. The method according to claim 17, wherein the acute exacerbation is acute exacerbation of chronic bronchitis in a human with COPD.

20. The method according to claim 17, wherein the acute exacerbation is acute bacterial exacerbation of chronic bronchitis in a human with COPD.

21. The method according to claim 17 wherein said formulation comprises about 4 parts by weight of fosfomycin to about 1 parts by weight of tobramycin.

22. The method according to claim 17, wherein said formulation is in a pharmaceutically acceptable solution and is suitable for administration by a nebulizer.

23. The method according to claim 17, wherein said formulation is a dry powder suitable for administration by a dry powder inhaler or metered dose inhaler.

24. The method according to claim 17, wherein said formulation consists of from about 1 to about 200 mg of fosfomycin and from about 0.1 to about 86 mg of tobramycin.

25. A method for reducing the frequency, severity or duration of one or more symptoms of acute exacerbation of COPD in a human, comprising administering by inhalation to said human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

26. The method according to claim 25, wherein said one or more symptoms are selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion.

27. The method according to claim 25, wherein the acute exacerbation is acute exacerbation of chronic bronchitis in a human with COPD.

28. The method according to claim 25, wherein the acute exacerbation is acute bacterial exacerbation of chronic bronchitis in a human with COPD.

29. The method according to claim 25 wherein said formulation comprises about 4 parts by weight of fosfomycin to about 1 parts by weight of tobramycin.

30. The method according to claim 25, wherein said formulation is in a pharmaceutically acceptable solution and is suitable for administration by a nebulizer.

31. The method according to claim 25, wherein said formulation is a dry powder suitable for administration by a dry powder inhaler or metered dose inhaler.

32. The method according to claim 25, wherein said formulation consists of from about 1 to about 200 mg of fosfomycin and from about 0.1 to about 86 mg of tobramycin.

33. A method for treating pulmonary inflammation in a human with COPD comprising administering by inhalation to said human an aerosol formulation comprising an effective amount of a combination of fosfomycin and tobramycin, wherein the weight ratio is from about 7 to about 9 parts by weight of fosfomycin to from about 1 to about 3 parts by weight of tobramycin.

34. A method of treating a bacterial infection in the respiratory tract of a human by administering by inhalation to said human an aerosol formulation consisting of fosfomycin and tobramycin and optionally one or more pharmaceutically acceptable carriers, excipients and/or diluents, wherein the weight ratio is from about 7 to about 9 parts by weight of fosfomycin to from about 1 to about 3 parts by weight of tobramycin, wherein said formulation is suitable for administration by a nebulizer, dry powder inhaler or metered dose inhaler
the improvement comprising
reducing the frequency, severity or duration of acute exacerbation in a human with COPD.

35. The method according to claim 34, the improvement comprising reducing the frequency, severity or duration of acute exacerbations of chronic bronchitis in a human with COPD.

36. The method according to claim 34, the improvement comprising reducing the frequency, severity or duration of acute bacterial exacerbations of chronic bronchitis in a human with COPD.
